(19) United States
(12) Patent Application Publication
(10) Pub. No.: US 2007/0218022 A1
(43) Pub. Date: Sep. 20, 2007

(54) Photostable sunscreen compositions comprising cinnamic acid ester UV-B filters, dibenzoylmethane UV-A filters and s-triazine compounds

(75) Inventors: Herve Richard, Les Pavillons Sous Bois (FR); Didier Candau, Bievres (FR)

Correspondence Address: BUCHANAN, INGERSOLL & ROONEY PC, POST OFFICE BOX 1404, ALEXANDRIA, VA, 22313-1404, US

(73) Assignee: L'OREAL, PARIS, FRANCE

(21) Appl. No.: 11/702,070

(22) Filed: Feb. 5, 2007

Related U.S. Application Data
(60) Provisional application No. 60/773,659, filed on Feb. 16, 2006.

(30) Foreign Application Priority Data
Feb. 3, 2006 (FR) 06/50390

(51) Int. Cl. A61K 8/49; A61K 8/36

(52) U.S. Cl. 424/59

(57) ABSTRACT

Photostable sunscreen compositions containing at least one cinnamic acid ester UV-B screening agent, at least one dibenzoylmethane compound UV-A screening agent and at least one particular s-triazine compound have improved effectiveness with respect to UV-B radiation, while maintaining the balance in UV-A/UV-B protection during exposure to the sun.

### CROSS-REFERENCE TO PRIORITY/PROVISIONAL APPLICATIONS

This application claims priority under 35 U.S.C. § 119 of FR 06/50390, filed Feb. 3, 2006, and of U.S. Provisional Application No. 60/773,659, Feb. 16, 2006, each hereby expressly incorporated by reference and each assigned to the assignee hereof.

### CROSS-REFERENCE TO COMPANION APPLICATIONS

Copending applications Serial No. [Attorney Docket No. 1016800-000796] and Serial No. [Attorney Docket No. 1016800-000797], filed concurrently herewith and each also assigned to the assignee hereof.

### BACKGROUND OF THE INVENTION

1. Technical Field of the Invention

The present invention relates to novel sunscreen compositions, in particular cosmetic sunscreen compositions for topical application, comprising the combination of a cinnamic acid ester UV-B filter, of a dibenzoylmethane compound UV-A filter and of an s-triazine having two hindered para-aminobenzalmalonate substituents and a para-aminobenzoate substituent having the specific formula (I) below.

The present invention also relates to a method for photostabilizing with respect to UV radiation at least one cinnamic acid ester UV-B screening agent in the presence of a dibenzoylmethane compound UV-A screening agent by combining said mixture of screening agents with at least one s-triazine compound having two hindered para-aminobenzalmalonate substituents and a para-aminobenzoate substituent of specific formula (I).

This invention also relates to the formulation of such s-triazine compound into compositions comprising, in a cosmetically acceptable vehicle, the combination of at least one cinnamic acid ester UV-B screening agent and at least one dibenzoylmethane compound screening agent for the purpose of improving the effectiveness of such compositions with respect to UV-B rays and of maintaining the balance in UV-A/UV-B protection during member exposure to the sun.

2. Description of Background and/or Related and/or Prior Art

It is known that light radiation with wavelengths ranging from 280 nm to 400 nm makes possible browning of the human epidermis and that rays with wavelengths more particularly of from 280 to 320 nm, known as UV-B radiation, causes erythemas and skin burns which may be harmful to the development of natural tanning. For these reasons and for aesthetic reasons, there exists a constant demand for means for controlling this natural tanning for the purpose of thus controlling the color of the skin; it is thus advisable to screen out this UV-B radiation.

It is also known that UV-A rays, with wavelengths of from 320 to 400 nm, which cause browning of the skin, are capable of bringing about a detrimental change in the latter, in particular, in the case of sensitive skin or of skin continually exposed to solar radiation. UV-A rays cause, in particular, a loss of elasticity of the skin and the appearance of wrinkles, resulting in premature cutaneous aging. They promote the triggering member of the erythemal reaction or accentuate this reaction in certain individuals and can even be the cause of phototoxic or photoallergic reactions. Thus, for aesthetic and cosmetic reasons, such as the retention of the natural elasticity of the skin, for example, individuals increasingly desire to control the effects of UV-A rays on their skin. It is thus desirable also to screen out UV-A radiation.

For the purpose of providing protection of the skin and keratinous substances against UV radiation, anti-sun/sunscreen compositions comprising organic screening agents which are active in the UV-A region and which are also active in the UV-B region are generally applied. The majority of these screening agents are fat-soluble.

In this regard, a particularly advantageous family of UV-B screening agents currently comprises cinnamic acid esters and in particular, 2-ethylhexyl para-methoxycinnamate, this being because these exhibit a high intrinsic absorbency. These cinnamic acid ester compounds, which are now per se well known as screening agents active in the UV-B region, are disclosed in particular, in FR-A-2,315,908; furthermore, 2-ethylhexyl para-methoxycinnamate is currently commercially available under the trademark “Parsol MCX” by DSM.

Also in this regard, a particularly advantageous family of UV-A screening agents currently comprises dibenzoylmethane compounds and in particular, 4-(tert-butyl)-4′-methoxydibenzoylmethane, because these exhibit a high intrinsic absorbency. These dibenzoylmethane compounds, which are now per se well known as screening agents active in the UV-A region, are disclosed, in particular, in FR-A-2,326,405 and FR-A-2,440,933, and also in EP-A-0,114,607; furthermore, 4-(tert-butyl)-4′-methoxydibenzoylmethane is currently commercially available under the trademark “Parsol 1789” by DSM.

The combination of these two families of screening agents is extremely advantageous in providing a broad and balanced screening system over the entire UV spectrum.

Unfortunately, it transpires that both cinnamic acid esters and dibenzoylmethane compounds are products which are relatively sensitive to ultraviolet radiation, that is to say, more specifically, that they exhibit an unfortunate tendency to decompose more or less rapidly under the action of the latter.

Furthermore, it also transpires that combining a dibenzoylmethane compound with a cinnamic acid ester imparts enhanced instability on the said cinnamic acid ester.

As the two types of screening agents are unstable by themselves and in combination, the amounts lost no longer make it possible to ensure good coverage of the UV band and the screening profile of the formulation may thus be unbalanced and excessively high exposure of the skin or hair to a portion of the UV spectrum may thus result. Thus, this substantial lack of stability of cinnamic acid esters in the presence of dibenzoylmethane compounds in the face of the UV radiation to which they are intended to be subjected does not make it possible to guarantee stable and constant protection during member prolonged exposure to sunlight.

Under these conditions, the quality of protection can be greatly affected by, on the one hand, an overall decrease in the level of protection and by, on the other hand, a major imbalance in screening in the UV-B region. In point of fact, while repeated applications at regular and close intervals of time can correct the decrease in the level of protection, no operation can compensate for the imbalance experienced by the screening profile of the formulation.

### SUMMARY OF THE INVENTION

A novel family of s-triazine compounds of formula (I) below has now been discovered, having two substituents selected from among hindered para-aminobenzalmalonate and para-aminobenzalmalonamide substituents and an aminobenzoate or aminobenzamide substituent, which make it possible to substantially improve the photochemical stability (or photostability) of the mixture of the cinnamic acid compounds and dibenzoylmethane compounds, thus ensuring the same UV-B/UV-A balance throughout exposure to the sun.

This discovery is the basis of the present invention.

Thus, the present invention features compositions comprising, formulated into a cosmetically acceptable vehicle, at least one UV screening system, which comprises:

(a) at least one cinnamic acid ester UV-B screening agent,

(b) at least one dibenzoylmethane compound UV-A screening agent, and

(c) at least one s-triazine compound of formula (I) below.

The present invention also features a method for improving the chemical stability with respect to UV radiation of at least one cinnamic acid ester UV-B screening agent in the presence of at least one dibenzoylmethane compound UV-A screening agent, which comprises combining, with this mixture of UV screening agents, at least one s-triazine compound of formula (I) below.

This invention also features formulating an s-triazine compound of formula (I) below, into a composition comprising, in a cosmetically acceptable vehicle, at least one cinnamic acid ester UV-B screening agent and at least one dibenzoylmethane compound for improving the chemical stability of the said UV-B screening agent with respect to UV radiation.

The present invention also features formulating an s-triazine compound of formula (I) below, into a composition comprising, in a cosmetically acceptable vehicle, at least one cinnamic acid ester UV-B screening agent and at least one dibenzoylmethane compound for improving the effectiveness of the said composition with respect to UV-B rays.

The present invention also features formulating an s-triazine compound of formula (I) below, into a composition comprising, in a cosmetically acceptable vehicle, at least one cinnamic acid ester UV-B screening agent and at least one dibenzoylmethane compound UV-A screening agent for maintaining the balance in UV-A/UV-B protection during exposure to the sun.

Other characteristics, aspects and advantages of the invention will become apparent from the detailed description which will follow.

In the present description, the term “system which screens out UV radiation” means an agent which screens out UV radiation composed either of a single organic or inorganic compound which screens out UV radiation or of a mixture of several organic or inorganic compounds which screen out UV radiation, for example a mixture comprising a UV-A screening agent and a UV-B screening agent.

The term “cosmetically acceptable” means compatible with the skin and/or its superficial body growths, which exhibits a pleasant color, a pleasant smell and a pleasant feel, and which does not cause unacceptable discomfort (smarting, tightness, red blotches) apt to dissuade the consumer from using this composition.

### DETAILED DESCRIPTION OF BEST MODE AND SPECIFIC/PREFERRED EMBODIMENTS OF THE INVENTION

The cinnamic acid esters according to the invention are preferably selected from among those having the following formula (A): 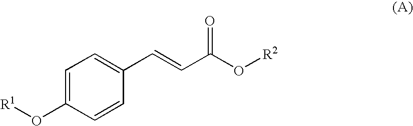
in which:

the radicals R¹ and R² are each, independently of one another, a linear or branched C₁-C₂₄ alkyl radical and more particularly a linear or branched C₁-C₈ alkyl radical, such as methyl, ethyl, propyl, isobutyl, butyl, sec-butyl, isobutyl, pentyl, neopentyl, hexyl and 2-ethylhexyl.

Exemplary cinnamic acid ester compounds of formula (A) include:
Ethylhexyl Methoxycinnamate or Cinoxate, marketed in particular, under the trademark “Parsol MCX” by DSM,
Isopropyl Methoxycinnamate,
Isoamyl Methoxycinnamate, marketed under the trademark “Neo Heliopan E 1000” by Symrise,
Diisopropyl Methylcinnamate.

The cinnamic acid ester or esters are present in the compositions according to the invention at contents which preferably range from 0.01% to 20% by weight, more preferably from 0.1% to 10% by weight and more preferably still from 0.1% to 8% by weight, with respect to the total weight of the composition.

Exemplary dibenzoylmethane compounds include:
2-methyldibenzoylmethane,
4-methyidibenzoylmethane,
4-isopropyldibenzoylmethane,
4-(tert-butyl)dibenzoylmethane,
2,4-dimethyldibenzoylmethane,
2,5-dimethyldibenzoylmethane,
4,4′-diisopropyldibenzoylmethane,
4,4′-dimethoxydibenzoylmethane,
4-(tert-butyl)-4′-methoxydibenzoylmethane,
2-methyl-5-isopropyl-4′-methoxydibenzoylmethane,
2-methyl-5-(tert-butyl)-4′-methoxydibenzoylmethane,
2,4-dimethyl-4′-methoxydibenzoylmethane,
2,6-dimethyl-4-(tert-butyl)-4′-methoxydibenzoylmethane.

Preferred among the dibenzoylmethane compounds indicated is 4-isopropyldibenzoylmethane, marketed under the trademark “Eusolex 8020” by Merck and having the following formula:

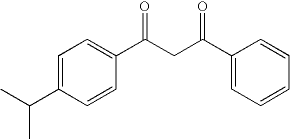

Very particularly preferred is 4-(tert-butyl)-4′-methoxydibenzoylmethane or Butyl Methoxy Dibenzoylmethane, marketed under the trademark “Parsol 1789” by DSM; this screening agent has the following formula:

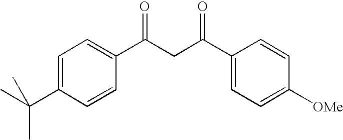

The dibenzoylmethane compound or compounds are present in the compositions in accordance with the invention at contents which preferably range from 0.01% to 20% by weight, more preferably from 0.1% to 10% by weight and more preferably still from 0.1% to 6% by weight, with respect to the total weight of the composition.

The s-triazine compounds in accordance with the present invention have the following general formula (I): 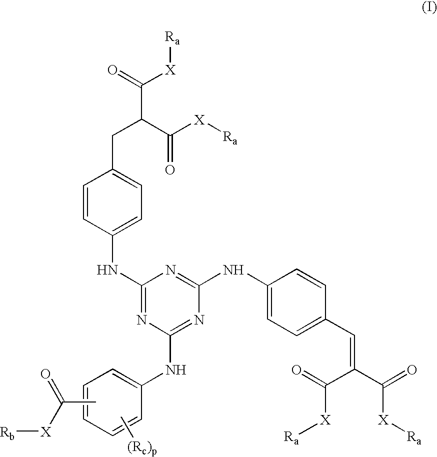
in which:

the groups X, which may be identical or different, are each —O— or —NR₆—;

the radicals Rₐ, which may be identical or different, are each a radical of formula (II): 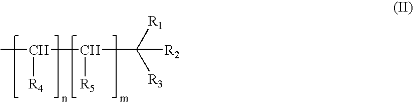
in which:

the radicals R₁ and R₂, which may be identical or different, are each a linear or branched C₁-C₈ alkyl radical; with the proviso that R₁ and R₂ may together form a C₅-C₈ ring member, optionally substituted by 1, 2 or 3 linear or branched C₁-C₄ alkyl radicals;

the radicals R₃, R₄ and R₅, which may be identical or different, are each a hydrogen atom or a linear or branched C₁-C₄ alkyl radical;

n has the value 0 or 1;

m has the value 0 or 1;

with the provisos that:

(i) when n=1 and R₄ is hydrogen, then m is equal to 0 and R₃ is other than hydrogen; and

(ii) when R₁ and R₂ together form a C₅-C₈ ring member, then the sum n+m is other than 2;

R₆ is hydrogen or a C₁-C₈ alkyl radical;

R_{b} is a linear or branched and optionally unsaturated C₁-C₂₀ alkyl radical, a C₅-C₁₂ cycloalkyl radical optionally substituted by 1 to 3 linear or branched C₁-C₄ alkyl radicals, the —(CH₂CHR₇—O)_{q}R₈ radical or the —CH₂—CH(OH)—CH₂—O—R₈ radical;

R₇ is hydrogen or methyl;

R₈ is hydrogen or a C₁-C₈ alkyl radical;

q=1-20; with the further proviso that the COXR_{b} group can be in the ortho, meta or para position with respect to the amino group;

R_{c} is a saturated or unsaturated and linear or branched C₁-C₂₀ alkyl radical, the OH group or a linear or branched C₁-C₂₀ alkoxy radical; and

p is equal to 0, 1 or 2.

In the above formula (I), the alkyl radicals can be selected in particular, from among the methyl, ethyl, n-propyl, isopropyl, n-butyl, isobutyl, tert-butyl, n-amyl, isoamyl, neopentyl, n-hexyl, n-heptyl, n-octyl, 2-ethylhexyl and tert-octyl radicals. The alkyl radical which is particularly preferred is the methyl radical.

The cycloalkyl radicals can be selected in particular from among the cyclopentyl, cyclohexyl and cycloheptyl radicals. The cycloalkyl radical which is particularly preferred is the cyclohexyl radical. These radicals can be substituted by C₁-C₄ alkyl radicals preferably selected from among methyl, isopropyl and tert-butyl.

Among the preferred compounds of formula (I), those for which the two following conditions are satisfied are more preferred:

(a) n=m=0 and

(b) R₁, R₂, R₃ are each a C₁-C₄ alkyl radical and more particularly methyl or else R₃ is hydrogen and R₁ and R₂ together form a C₅-C₈ ring member optionally substituted by one or two alkyl radicals and more particularly cyclohexyl.

Among the preferred compounds of formula (I), those for which the following two conditions are satisfied are also more preferred:

(a) n=1 and R₄ is an alkyl radical, in particular methyl, or m=1 and R₅ is an alkyl, in particular methyl, and

(b) R₁ and R₂ are each a C₁-C₄ alkyl radical and more particularly methyl.

Exemplary compounds of formula (I) which are more particularly preferred are those selected from among the compounds of following formulae (1) to (10):

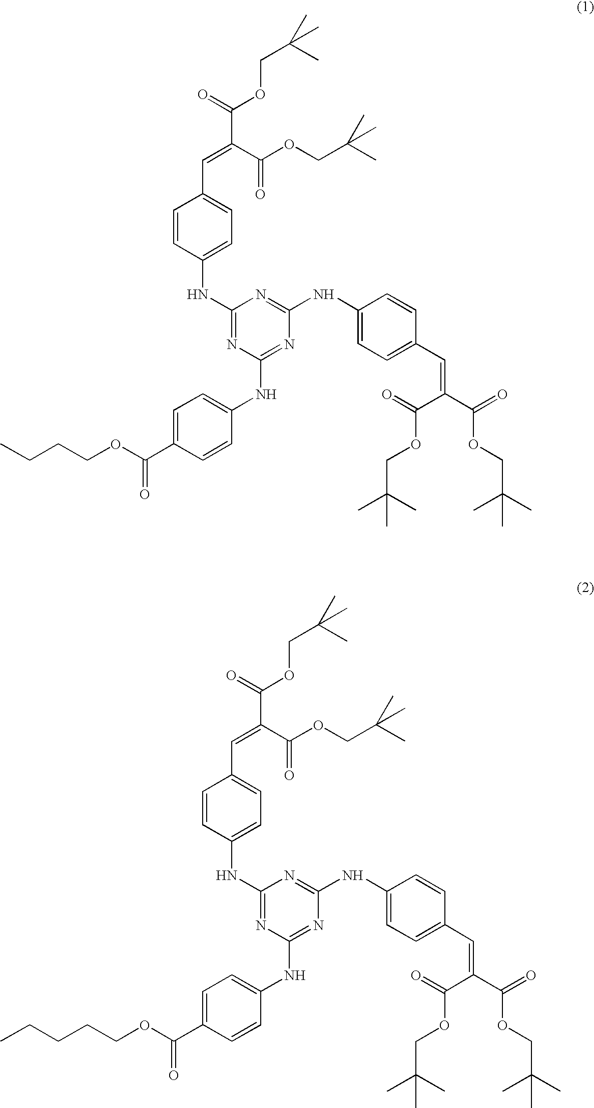

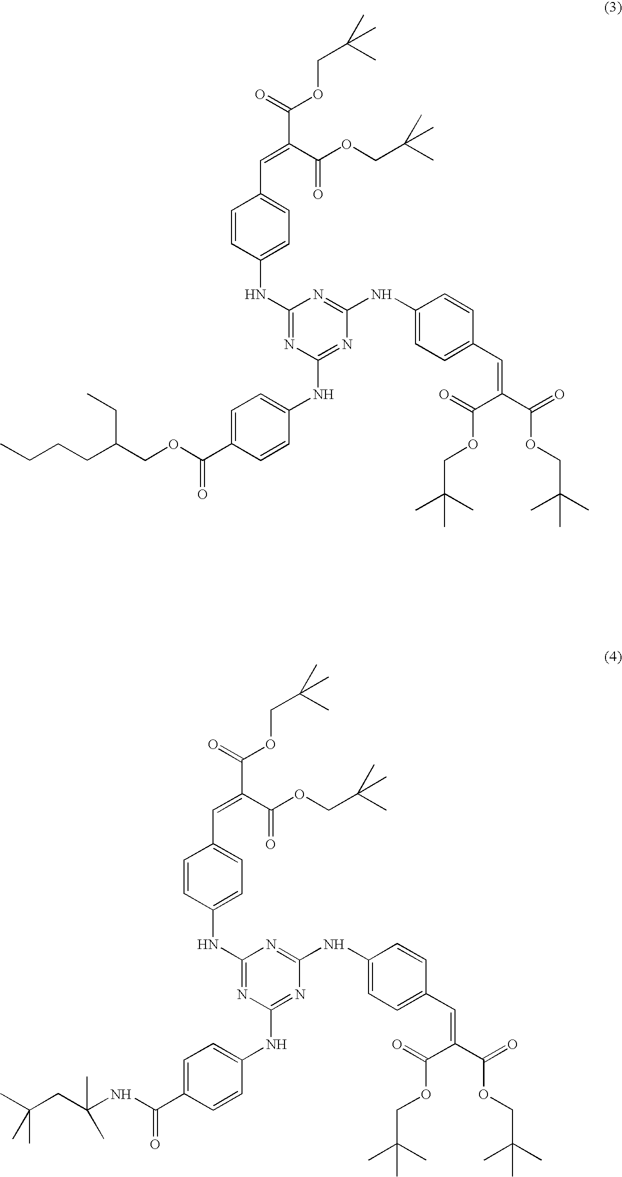

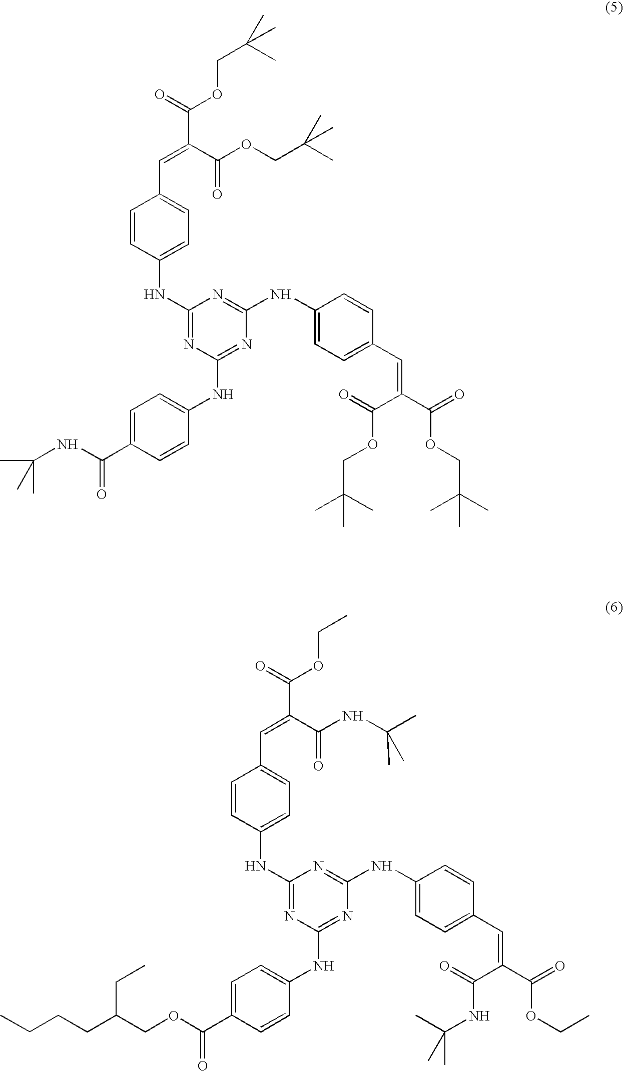

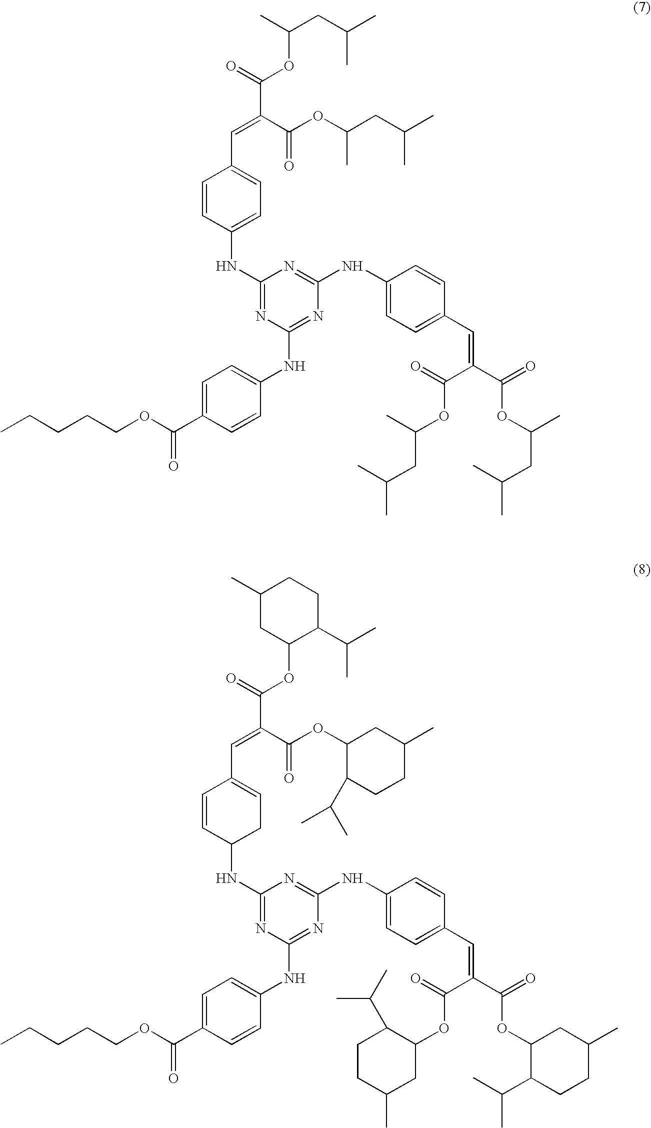

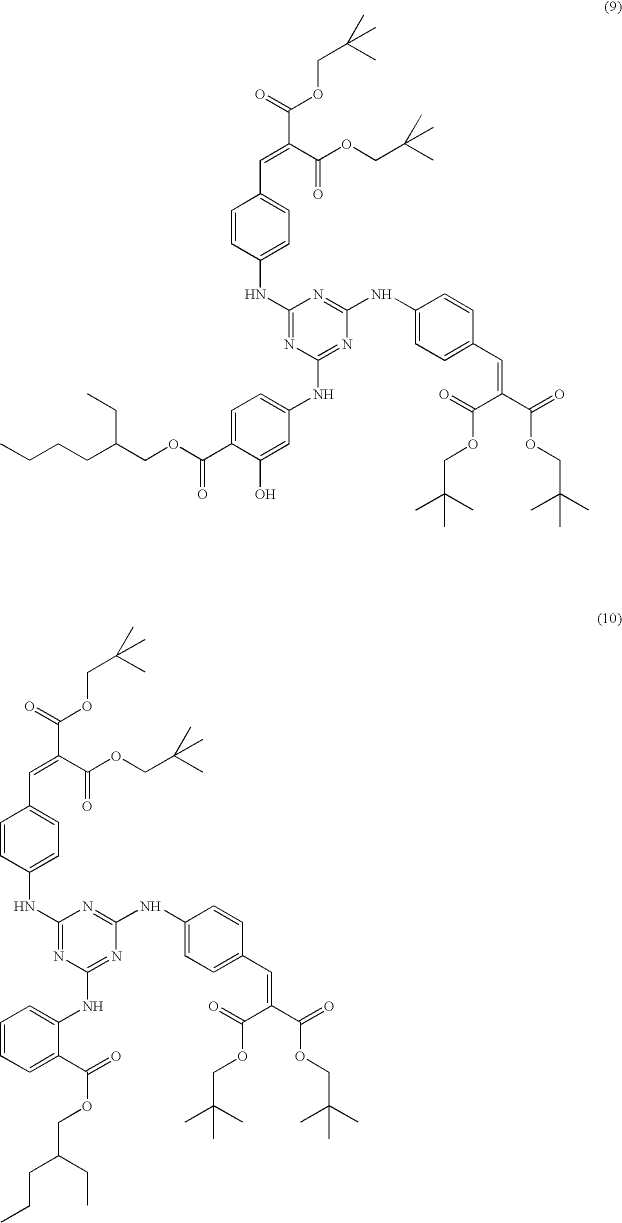

More particularly preferred, among these compounds, is 2,4-bis(dineopentyl 4′-aminobenzalmalonate)-6-(butyl 4″-aminobenzoate)-s-triazine of formula (1).

The compounds of formula (I) can be prepared according to the following scheme (A): 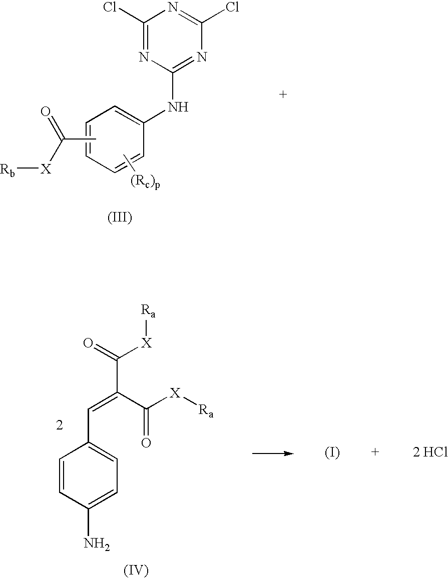
in which Rₐ, X, R_{b}, R_{c}, and p have the definitions of the above formula (I).

The above reactions can optionally be carried out in the presence of a solvent (for example: toluene, xylene or 1,2-dichloroethane), at a temperature of from 0° C. and 250° C., more particularly from 5° C. and 150° C. They can also be carried out using microwaves in the presence or absence of a solvent (for example: toluene, xylene or 1,2-dichloroethane) or in the presence or absence of 10% of graphite, at a temperature of 50° C. to 150° C., at a power of 50 to 150 watts, for a period of time of 10 to 30 minutes.

The compounds of formula (III) can be prepared according to known methods disclosed, for example, in EP-0,507,691 assigned to the assignee hereof.

The compounds of formula (I) are generally present in the compositions of the invention in proportions ranging from 0.01% to 20% by weight, preferably from 0.1% to 10% by weight, with respect to the total weight of the composition.

Furthermore, the compositions in accordance with the invention can comprise other additional organic or inorganic UV screening agents active in the UV-A and/or UV-B region which are water-soluble or fat-soluble or else insoluble in the cosmetic solvents commonly employed.

The additional organic screening agents are selected in particular from among anthranilates; salicylic derivatives; camphor derivatives; triazine derivatives other than those of the invention, such as those disclosed in U.S. Pat. No. 4,367,390, EP-863,145, EP-517,104, EP-570,838, EP-796,851, EP-775,698, EP-878,469, EP-933,376, EP-507,691, EP-507,692, EP-790,243 and EP-944,624; benzophenone derivatives; β,β-diphenylacrylate derivatives; benzotriazole derivatives; benzalmalonate derivatives other than those of the invention; benzimidazole derivatives; imidazolines; bis-benzoazolyl derivatives, as disclosed in EP-669,323 and U.S. Pat. No. 2,463,264; p-aminobenzoic acid (PABA) derivatives; methylenebis(hydroxyphenylbenzotriazole) derivatives, as disclosed in U.S. Pat. Nos. 5,237,071, 5,166,355, GB-2,303,549, DE-197,26,184 and EP-893,119; screening polymers and screening silicones, such as those disclosed in particular, in WO 93/04665; dimers derived from α-alkylstyrene, such as those disclosed in DE-198,55,649; 4,4-diarylbutadienes, as disclosed in EP-0,967,200, DE-197,46,654, DE-197,55,649, EP-A-1,008,586, EP-1,133,980 and EP-133,981; and mixtures thereof.

Exemplary additional organic screening agents are those denoted below under their INCl names:

Para-Aminobenzoic Acid Derivatives:
PABA,
Ethyl PABA,
Ethyl Dihydroxypropyl PABA,
Ethylhexyl Dimethyl PABA, marketed in particular, under the trademark “Escalol 507” by ISP,
Glyceryl PABA,
PEG-25 PABA, marketed under the trademark “Uvinul P25” by BASF.

Salicylic Derivatives:
Homosalate, marketed under the trademark “Eusolex HMS” by Rona/EM Industries,
Ethylhexyl Salicylate, marketed under the trademark “Neo Heliopan OS” by Haarmann and Reimer,
Dipropyleneglycol Salicylate, marketed under the trademark “Dipsal” by Scher,
TEA Salicylate, marketed under the trademark “Neo Heliopan TS” by Haarmann and Reimer.

β,β-Diphenylacrylate Derivatives:
Octocrylene, marketed in particular, under the trademark “Uvinul N539” by BASF,
Etocrylene, marketed in particular, under the trademark “Uvinul N35” by BASF.

Benzophenone Derivatives:
Benzophenone-1, marketed under the trademark “Uvinul 400” by BASF,
Benzophenone-2, marketed under the trademark “Uvinul D50” by BASF,
Benzophenone-3 or Oxybenzone, marketed under the trademark “Uvinul M40” by BASF,
Benzophenone-4, marketed under the trademark “Uvinul MS40” by BASF,
Benzophenone-5,
Benzophenone-6, marketed under the trademark “Helisorb 11” by Norquay,
Benzophenone-8, marketed under the trademark “Spectra-Sorb UV-24” by American Cyanamid,
Benzophenone-9, marketed under the trademark “Uvinul DS-49” by BASF, Benzophenone-1 2,
Diethylamino Hydroxybenzoyl Hexyl Benzoate, marketed under the trademark “Uvinul A Plus” by BASF.

Benzylidenecamphor Derivatives:
3-Benzylidene camphor, manufactured under the trademark “Mexoryl SD” by Chimex,
4-Methylbenzylidene camphor, marketed under the trademark “Eusolex 6300” by Merck,
Benzylidene Camphor Sulfonic Acid, manufactured under the trademark “Mexoryl SL” by Chimex,
Camphor Benzalkonium Methosulfate, manufactured under the trademark “Mexoryl SO” by Chimex,
Terephthalylidene Dicamphor Sulfonic Acid, manufactured under the trademark “Mexoryl SX” by Chimex,
Polyacrylamidomethyl Benzylidene Camphor, manufactured under the trademark “Mexoryl SW” by Chimex.

Phenylbenzimidazole Derivatives:
Phenylbenzimidazole Sulfonic Acid, marketed in particular, under the trademark “Eusolex 232” by Merck,
Disodium Phenyl Dibenzimidazole Tetrasulfonate, marketed under the trademark “Neo Heliopan AP” by Haarmann and Reimer.

Triazine Derivatives:
Bis-Ethylhexyloxyphenol Methoxyphenyl Triazine, marketed under the trademark “Tinosorb S” by Ciba-Geigy,
Ethylhexyl Triazone, marketed in particular, under the trademark “Uvinul T150” by BASF,
Diethylhexyl Butamido Triazone, marketed under the trademark “Uvasorb HEB” by Sigma 3V.

Phenylbenzotriazole Derivatives:
Drometrizole Trisiloxane, marketed under the trademark “Silatrizole” by Rhodia Chimie,
Methylene Bis-Benzotriazolyl Tetramethylbutylphenol, marketed in the solid form under the trademark “Mixxim BB/100” by Fairmount Chemical or in the micronized form in aqueous dispersion under the trademark “Tinosorb M” by Ciba Specialty Chemicals.

Anthranilic Derivatives:
Menthyl anthranilate, marketed under the trademark “Neo Heliopan MA” by Haarmann and Reimer.

Imidazoline Derivatives:
Ethylhexyl Dimethoxybenzylidene Dioxoimidazoline Propionate,

Benzalmalonate Derivatives:
Polyorganosiloxanes comprising benzalmalonate functional groups, such as Polysilicone-15, marketed under the trademark “Parsol SLX” by Hoffmann-LaRoche.

4,4-Diarylbutadiene Derivatives:
1,1 -Dicarboxy(2,2′-dimethylpropyl)-4,4-diphenylbutadiene, and mixtures thereof.

The preferred additional organic UV screening agents are selected from among:

Ethylhexyl Salicylate,

Homosalate,

Octocrylene,

Phenylbenzimidazole Sulfonic Acid,

Disodium Phenyl Dibenzimidazole Tetrasulfonate,

Benzophenone-3,

Benzophenone-4,

Benzophenone-5,

n-Hexyl 2-(4-diethylamino-2-hydroxybenzoyl)benzoate,

4-Methylbenzylidene Camphor,

Terephthalylidene Dicamphor Sulfonic Acid,

Anisotriazine,

Ethylhexyl Triazone,

Diethylhexyl Butamido Triazone,

Methylene Bis-Benzotriazolyl Tetramethylbutylphenol,

Drometrizole Trisiloxane,

Polysilicone-15,

1,1-Dicarboxy(2,2′-Dimethylpropyl)-4,4-Diphenylbutadiene,

and mixtures thereof.

The additional inorganic photoprotective agents are selected from among pigments and more preferably still pigments having a mean size of the primary particles generally ranging from 5 nm to 100 nm, preferably from 10 nm to 50 nm formed of metal oxides which may or may not be treated, such as, for example, pigments formed of titanium oxide (amorphous or crystalline in the rutile and/or anatase form), iron oxide, zinc oxide, zirconium oxide or cerium oxide.

The pigments treated are pigments which have been subjected to one or more surface treatments of a chemical, electronic, mechanochemical and/or mechanical nature with compounds such as described, for example, in Cosmetics & Toiletries, February 1990, Vol. 105, pp. 53-64, such as amino acids, beeswax, fatty acids, fatty alcohols, anionic surfactants, lecithins, sodium, potassium, zinc, iron, or aluminum salts of fatty acids, metal alkoxides (titanium or aluminum alkoxides), polyethylene, silicones, proteins (collagen, elastin), alkanolamines, silicon oxides, metal oxides, sodium hexametaphosphate, alumina or glycerol.

The treated pigments can more particularly be titanium oxides treated with:
silica and alumina, such as the products “Microtitanium Dioxide MT 500 SA” and “Microtitanium Dioxide MT 100 SA” from Tayca and the products “Tioveil Fin”, “Tioveil OP”, “Tioveil MOTG” and “Tioveil IPM” from Tioxide,
alumina and aluminum stearate, such as the product “Microtitanium Dioxide MT 100 T” from Tayca,
alumina and aluminum laurate, such as the product “Microtitanium Dioxide MT 100 S” from Tayca,
iron oxides and iron stearate, such as the product “Microtitanium Dioxide MT 100 F” from Tayca,
silica, alumina and silicone, such as the products “Microtitanium Dioxide MT 100 SAS”, “Microtitanium Dioxide MT 600 SAS” and “Microtitanium Dioxide MT 500 SAS” from Tayca,
sodium hexametaphosphate, such as the product “Microtitanium Dioxide MT 150 W” from Tayca,
octyltrimethoxysilane, such as the product “T-805” from Degussa,
alumina and stearic acid, such as the product “UVT-M160” from Kemira,
alumina and glycerol, such as the product “UVT-M212” from Kemira,
alumina and silicone, such as the product “UVT-M262” from Kemira.

Other titanium oxide pigments treated with a silicone are preferably TiO₂ treated with octyltrimethylsilane and for which the mean size of the individual particles is from 25 and 40 nm, such as that marketed under the trademark “T 805” by Degussa Silices, TiO₂ treated with a polydimethylsiloxane and for which the mean size of the individual particles is 21 nm, such as that marketed under the trademark “70250 Cardre UF TiO2SI3” by Cardre, anatase/rutile TiO₂ treated with a polydimethylhydrosiloxane and for which the mean size of the individual particles is 25 nm, such as that marketed under the trademark “Microtitanium Dioxyde USP Grade Hydrophobic” by Color Techniques.

The uncoated titanium oxide pigments are, for example, marketed by Tayca under the trademarks “Microtitanium Dioxide MT 500 B” or “Microtitanium Dioxide MT600 B”, by Degussa under the trademark “P 25”, by Wacker under the trademark “Oxyde de titane transparent PW”, by Miyoshi Kasei under the trademark “UFTR”, by Tomen under the trademark “ITS” and by Tioxide under the trademark “Tioveil AQ”.

The uncoated zinc oxide pigments are, for example:
those marketed under the trademark “Z-cote” by Sunsmart;
those marketed under the trademark “Nanox” by Elementis;
those marketed under the trademark “Nanogard WCD 2025” by Nanophase Technologies.

The coated zinc oxide pigments are, for example:
those marketed under the trademark “Oxide zinc CS-5” by Toshibi (ZnO coated with polymethylhydrosiloxane);
those marketed under the trademark “Nanogard Zinc Oxide FN” by Nanophase Technologies (as a 40% dispersion in Finsolv Tenn., C₁₂-C₁₅ alkyl benzoate);
those marketed under the trademark “Daitopersion Zn-30” and “Daitopersion Zn-50” by Daito (dispersions in oxyethylenated polydimethylsiloxane/cyclopolymethylsiloxane comprising 30% or 50% of zinc oxides coated with silica and polymethylhydrosiloxane);
those marketed under the trademark “NFD Ultrafine ZnO” by Daikin (ZnO coated with phosphate of perfluoroalkyl and copolymer based on perfluoroalkylethyl as a dispersion in cyclopentasiloxane);
those marketed under the trademark “SPD-Z1” by Shin-Etsu (ZnO coated with silicone-grafted acrylic polymer dispersed in cyclodimethylsiloxane);
those marketed under the trademark “Escalol Z100” by ISP (alumina-treated ZnO dispersed in the ethylhexyl methoxycinnamate/PVP-hexadecene copolymer/methicone mixture);
those marketed under the trademark “Fuji ZnO-SMS-10” by Fuji Pigment (ZnO coated with silica and polymethylsilsesquioxane);
those marketed under the trademark “Nanox Gel TN” by Elementis (ZnO dispersed at 55% in C₁₂-C₁₅ alkyl benzoate with hydroxystearic acid polycondensate).

The uncoated cerium oxide pigments are marketed under the trademark “Colloidal Cerium Oxide” by Rhône-Poulenc.

The uncoated iron oxide pigments are, for example, marketed by Arnaud under the trademarks “Nanogard WCD 2002 (FE 45B)”, “Nanogard Iron FE 45 BL AQ”, “Nanogard FE 45R AQ” or “Nanogard WCD 2006 (FE 45R)”, or by Mitsubishi under the trademark “TY-220”.

The coated iron oxide pigments are, for example, marketed by Arnaud under the trademarks “Nanogard WCD 2008 (FE 45B FN)”, “Nanogard WCD 2009 (FE 45B 556)”, “Nanogard FE 45 BL 345” or “Nanogard FE 45 BL” or by BASF under the trademark “Oxyde de fer transparent”.

Also exemplary are mixtures of metal oxides, in particular, of titanium dioxide and of cerium dioxide, including the mixture of equal weights of titanium dioxide coated with silica and of cerium dioxide coated with silica marketed by Ikeda under the trademark “Sunveil A”, and also the mixture of titanium dioxide and of zinc dioxide coated with alumina, with silica and with silicone, such as the product “M 261” marketed by Kemira, or coated with alumina, with silica and with glycerol, such as the product “M 211” marketed by Kemira.

The nanopigments can be introduced into the compositions according to the invention as is or in the pigment paste form, that is to say, as a mixture with a dispersant, as disclosed, for example, in the GB-A-2,206,339.

The additional photoprotective agents are generally present in the compositions according to the invention in proportions ranging from 0.01% to 20% by weight, with respect to the total weight of the composition, and preferably ranging from 0.1% to 10% by weight, with respect to the total weight of the composition.

The compositions according to the invention can also comprise agents for the artificial tanning and/or browning of the skin (self-tanning agents) and more particularly dihydroxyacetone (DHA). They are preferably present in amounts ranging from 0.1% to 10% by weight, with respect to the total weight of the composition.

The compositions in accordance with the present invention can additionally comprise conventional cosmetic adjuvants selected in particular, from among fatty substances, organic solvents, ionic or nonionic and hydrophilic or lipophilic thickeners, softening agents, humectants, opacifiers, stabilizing agents, emollients, silicones, anti-foaming agents, fragrances, preservatives, anionic, cationic, nonionic, zwitterionic or amphoteric surfactants, active principles, fillers, polymers, propellants, basifying or acidifying agents or any other ingredient commonly employed in the cosmetics and/or dermatological field.

The fatty substances can be an oil or a wax or their mixtures. The term “oil” means a compound which is liquid at ambient temperature. The term “wax” means a compound which is solid or substantially solid at ambient temperature and which has a melting point generally of greater than 35° C.

Exemplary oils are mineral oils (liquid paraffin); vegetable oils (sweet almond, macadamia, blackcurrant seed or jojoba oil); synthetic oils, such as perhydrosqualene, fatty alcohols, acids or esters (such as C₁₂-C₁₅ alkyl benzoate, marketed under the trademark “Finsolv TN” by Witco, octyl palmitate, isopropyl lanolate or triglycerides, including those of capric/caprylic acids), or oxyethylenated or oxypropylenated fatty esters and ethers; silicone oils (cyclomethicone, polydimethylsiloxanes or PDMSs); fluorinated oils; or polyalkylenes.

Exemplary waxy compounds include paraffin wax, carnauba wax, beeswax or hydrogenated castor oil.

Exemplary organic solvents include the lower alcohols and polyols. The latter can be selected from among glycols and glycol ethers, such as ethylene glycol, propylene glycol, butylene glycol, dipropylene glycol or diethylene glycol.

Exemplary hydrophilic thickeners include carboxyvinyl polymers, such as the Carbopols (Carbomers) and the Pemulens (acrylate/C₁₀-C₃₀-alkyl acrylate copolymer); polyacrylamides, such as, for example, the crosslinked copolymers marketed under the trademarks Sepigel 305 (CTFA name: polyacrylamide/C13-14 isoparaffin/Laureth 7) or Simulgel 600 (CTFA name: acrylamide/sodium acryloyldimethyltaurate copolymer/isohexadecane/polysorbate 80) by Seppic; optionally crosslinked and/or neutralized polymers and copolymers of 2-acrylamido-2-methylpropanesulfonic acid, such as the poly(2-acrylamido-2-methylpropanesulfonic acid) marketed by Hoechst under the trademark “Hostacerin AMPS” (CTFA name: ammonium polyacryldimethyltauramide); cellulose derivatives, such as hydroxyethylcellulose; polysaccharides and in particular, gums, such as xanthan gum; and their mixtures.

Exemplary lipophilic thickeners are the modified clays, such as hectorite and its derivatives, such as the products marketed under the Bentone trademarks.

Exemplary bioactive principles include:
agents for combating pollution and/or agents for combating free radicals;
depigmenting agents and/or propigmenting agents;
anti-glycation agents;
NO-synthase inhibitors;
agents which stimulate the synthesis of dermal or epidermal macromolecules and/or which prevent their decomposition;
agents which stimulate the proliferation of fibroblasts;
agents which stimulate the proliferation of keratinocytes;
muscle-relaxing agents;
tightening agents;
desquamating agents;
moisturizing agents;
anti-inflammatory agents;
agents which act on the energy metabolism of the cells;
insect repellents;
substance P or substance CRGP antagonists.

Of course, one skilled in this art will take care to select the additional optional compound or compounds mentioned above and/or their amounts so that the advantageous properties intrinsically associated with the compositions in accordance with the invention are not, or not substantially, detrimentally affected by the envisaged addition or additions.

The compositions according to the invention are formulated according to techniques well known to this art, in particular, those intended for the preparation of emulsions of oil-in-water or water-in-oil type. They can in particular, be provided in the form of a simple or complex (O/W, W/O, O/W/O or W/O/W) emulsion, such as a cream or a milk, or in the form of a gel or of a cream gel, in the form of a lotion, of a powder or of a solid stick, and can optionally be packaged as an aerosol and be provided in the foam or spray form.

Preferably, the compositions according to the invention are provided in the form of an oil-in-water or water-in-oil emulsion.

The emulsions generally comprise at least one emulsifier selected from among amphoteric, anionic, cationic or nonionic emulsifiers, used alone or as a mixture. The emulsifiers are appropriately selected according to the emulsion to be obtained (W/O or O/W emulsion).

Exemplary emulsifying surfactants which can be employed for the preparation of the W/O emulsions, for example, include sorbitan, glycerol or sugar alkyl esters or ethers; silicone surfactants, such as dimethicone copolyols, for example the mixture of cyclomethicone and of dimethicone copolyol marketed under the trademark “DC 5225 C” by Dow Corning, and alkyl dimethicone copolyols, such as lauryl methicone copolyol, marketed under the trademark “Dow Corning 5200 Formulation Aid” by Dow Corning, cetyl dimethicone copolyol, such as the product marketed under the trademark Abil EM 90R by Goldschmidt, and the mixture of cetyl dimethicone copolyol, of polyglycerol (4 mol) isostearate and of hexyl laurate marketed under the trademark Abil WE O9 by Goldschmidt. It is also possible to add thereto one or more coemulsifiers which, advantageously, can be selected from the group consisting of polyol alkyl esters. In particular, exemplary are polyol alkyl esters, esters of glycerol and/or of sorbitan, for example polyglycerol isostearate, such as the product marketed under the trademark Isolan GI 34 by Goldschmidt, sorbitan isostearate, such as the product marketed under the trademark Arlacel 987 by ICI, glycerol sorbitan isostearate, such as the product marketed under the trademark Arlacel 986 by ICI, and their mixtures.

For the O/W emulsions, exemplary emulsifiers are the nonionic emulsifiers, such as oxyalkylenated (more particularly polyoxyethylenated) esters of fatty acids and of glycerol; oxyalkylenated esters of fatty acids and sorbitan; oxyalkylenated (oxyethylenated and/or oxypropylenated) fatty acid esters; oxyalkylenated (oxyethylenated and/or oxypropylenated) fatty alcohol ethers; sugar esters, such as sucrose stearate; ethers of fatty alcohol and of sugar, in particular, alkyl polyglucosides (APG), such as decyl glucoside and lauryl glucoside, for example marketed by Henkel under the respective trademarks Plantaren 2000 and Plantaren 1200, cetearyl glucoside, optionally as a mixture with cetearyl alcohol, for example marketed under the trademark Montanov 68 by Seppic, under the trademark Tegocare CG90 by Goldschmidt and under the trademark Emulgade KE3302 by Henkel, and arachidyl glucoside, for example in the form of the mixture of arachidyl and behenyl alcohols and of arachidyl glucoside marketed under the trademark Montanov 202 by Seppic. According to a specific embodiment of the invention, the mixture of the alkyl polyglucoside as defined above with the corresponding fatty alcohol can be in the form of a self-emulsifying composition, for example as disclosed in WO-A-92/06778.

When an emulsion is involved, the aqueous phase of the latter can comprise a nonionic vesicular dispersion prepared according to known methods (Bangham, Standish and Watkins, J. Mol. Biol., 13, 238 (1965), FR-2,315,991 and FR-2,416,008).

The compositions according to the invention have applications in a large number of treatments, whether regime or regimen, in particular, cosmetic treatments, of the skin, lips and hair, including the scalp, in particular, for protecting and/or caring for the skin, lips and/or hair and/or for making up the skin and/or lips.

The present invention also features the use of the compositions according to the invention as defined above in the manufacture of products for the cosmetic treatment of the skin, lips, nails, hair, eyelashes, eyebrows and/or scalp, in particular, of care products and makeup products.

The cosmetic compositions according to the invention are useful, for example, as care product and/or sun protection product for the face and/or body with a liquid to semi-liquid consistency, such as milks, relatively smooth creams, cream gels or pastes. They can optionally be packaged in an aerosol and be provided in the foam or spray form.

The compositions according to the invention in the form of. vaporizable fluid lotions are applied to the skin or hair in the form of fine particles by means of pressurizing devices. The devices in accordance with the invention are well known to those skilled in this art and comprise non-aerosol pumps or “atomizers”, aerosol containers comprising a propellant and aerosol pumps using compressed air as propellant. The latter are disclosed in U.S. Pat. Nos. 4,077,441 and 4,850,517.

The compositions packaged in an aerosol in accordance with the invention generally comprise conventional propellants, such as, for example, hydrofluorinated compounds, dichlorodifluoromethane, difluoroethane, dimethyl ether, isobutane, n-butane, propane or trichlorofluoromethane. They are preferably present in amounts ranging from 15% to 50% by weight, with respect to the total weight of the composition.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLE 1

### Synthesis of 2,4-bis(dineopentyl 4′-aminobenzalmalonate)-6-(butyl 4“-aminobenzoate)-s-triazine of formula (1)

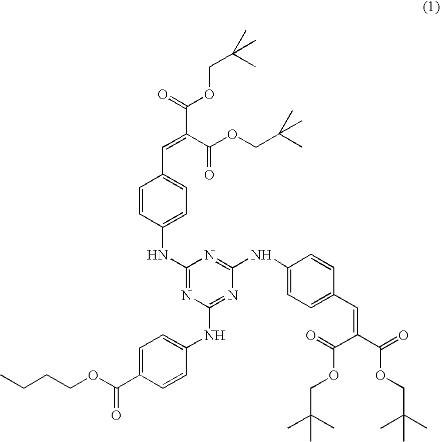

### First Stage: Preparation of 2,4-dichloro-6-(butyl 4′-aminobenzoate)-s-triazine

Cyanuryl chloride (20.7 g, 0.112 mol) is dissolved at 0-5° C. in 250 ml of acetone in a reactor. A solution of butyl para-aminobenzoate (21.7 g, 0.112 mol) dissolved in 70 ml of acetone is added thereto dropwise at 0° C.-5° C. over 1 hour. Subsequently, sodium bicarbonate (9.4 g, 0.112 mol) dissolved in 70 ml of water is added thereto. The heterogeneous mixture is left at a temperature of 0° C.-5° C. for 2 hours. The precipitate formed is filtered off and then washed with water and with acetone. After drying under vacuum, 37.2 g (97% yield) of 2,4-dichloro-6-(butyl 4′-aminobenzoate)-s-triazine are obtained in the form of a white powder:
UV (ethanol/DMSO): λₘₐₓ=298 nm, E1%=940,
   which is used as is in the following stage.

### Second Stage: Preparation of the Compound of Example 1

A mixture of the preceding product (7.41 g, 0.0213 mol) and of dineopentyl para-aminobenzalmalonate (14.66 g, 0.0422 mol) in suspension in 60 ml of toluene is heated at reflux for 7 hours 30 minutes while sparging with nitrogen. The mixture is cooled and dichloromethane is added. The organic phase is washed with a saturated sodium bicarbonate solution and then with water. The organic phase is dried and then concentrated under reduced pressure. The orange-colored oil obtained (17.8 g) is subjected to separation on a silica column (eluent: heptane/EtOAc 85:15). Clean fractions are recovered in the form of pale yellow flakes of the derivative of Example 1 (8.72 g, 43% yield):
UV (ethanol): λ=370 nm, E1%=623; λₘₐₓ=347 nm, E1%=847; λ=300 nm, E1%=432.

### EXAMPLE 2

### Synthesis of 2,4-bis(dineopentyl 4′-aminobenzalmalonate)-6-(amyl 4″-aminobenzoate)-s-triazine of formula (2)

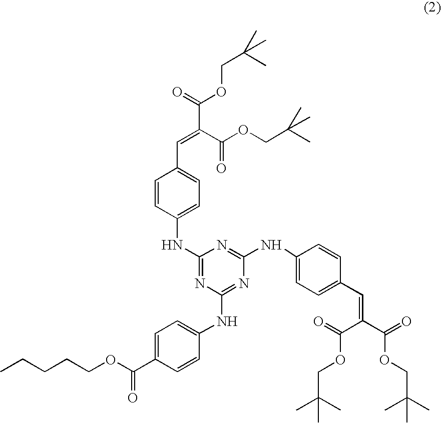

### First Stage: Preparation of 2,4-dichloro-6-(amyl 4′-aminobenzoate)-s-triazine

Cyanuryl chloride (14.7 g, 0.0796 mol) is dissolved in 200 ml of dioxane at 10° C. in a reactor. A solution of amyl para-aminobenzoate (16.5 g, 0.0796 mol) dissolved in 60 ml of dioxane and a solution of potassium carbonate (5.5 g, 0.0398 mol) dissolved in 30 ml of water are simultaneously added thereto dropwise at 10° C. over 1 hour. The heterogeneous mixture is left at a temperature of 10° C. for 2 hours. Approximately 300 ml of water are added and the precipitate formed is filtered off and then washed with water. After drying under vacuum, 26.4 g (93% yield) of 2,4-dichloro-6-(amyl 4′-aminobenzoate)-s-triazine are obtained in the form of a white powder used as is in the following stage.

### Second Stage: Preparation of the Compound of Example 2

The intimately mixed mixture of the preceding product (0.103 g, 0.29×10⁻³ mol), of dineopentyl para-aminobenzalmalonate (0.2 g, 0.58×10⁻³ mol) and of sodium bicarbonate (0.049 g, 0.58×10⁻³ mol) is left in a CEM Discover microwave system for 10 minutes at a temperature of 150° C. and under a power of 150 watts. The amorphous solid form is extracted with dichloromethane. The organic phase is washed 3 times with water, is dried and is then concentrated under reduced pressure. The yellow oil obtained is subjected to separation on a silica column (eluent:heptane/EtOAc 80:20). Clean fractions are recovered in the form of a pale yellow paste of the derivative of Example 2 (36 mg, 15% yield):
UV (ethanol): λ=375 nm, E1%=610; λₘₐₓ=347 nm, E1%=834; λ=300 nm, E1%=425.

### EXAMPLE 3

### Synthesis of 2,4-bis(dineopentyl 4′-aminobenzalmalonate)-6-(2-ethylhexyl 4″-aminobenzoate)-s-triazine of formula (3)

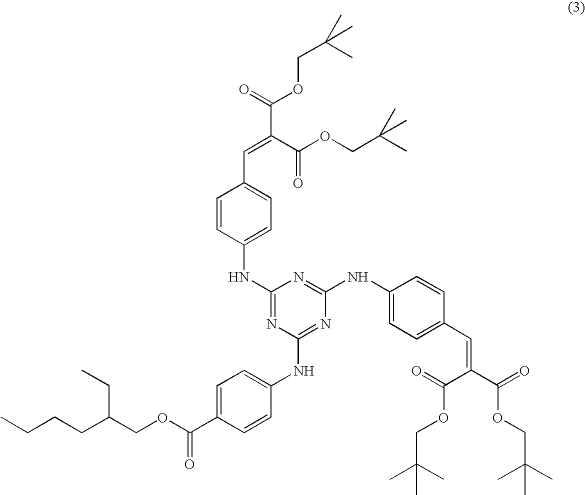

### First Stage: Preparation of 2,4-dichloro-6-(2-ethylhexyl 4′-aminobenzoate)-s-triazine

Cyanuryl chloride (18.4 g, 0.1 mol) is dissolved at 0° C.-5° C. in 150 ml of acetone in a reactor. Sodium bicarbonate (10.6 g, 0.1 mol) is added thereto and then a solution of 2-ethylhexyl para-aminobenzoate (24.9 g, 0.1 mol) dissolved in 150 ml of acetone is added dropwise at a temperature of less than 10° C. over 10 minutes. Subsequently, the heterogeneous mixture is left at laboratory temperature for 3 hours. 500 ml of water are run in. The precipitate formed is filtered off and then washed with water. After drying under vacuum, 38 g of an off-white solid are obtained. After recrystallizing this solid from 1,2-dichloroethane, 25.2 g (63% yield) of 2,4-dichloro-6-(2-ethylhexyl 4′-aminobenzoate)-s-triazine are obtained in the form of a white powder:
UV (ethanol/DMSO): λₘₐₓ=291 nm, E1%=732, which is used as is in the following stage.

### Second Stage: Preparation of the Compound of Example 3

A mixture of the preceding product (1.14 g, 2.87×10⁻³ mol) and of dineopentyl para-aminobenzalmalonate (2.2 g, 6.33×10⁻³ mol) in suspension in 35 ml of toluene is heated at reflux for 10 hours 30 minutes while sparging with nitrogen. The mixture is cooled and dichloromethane is added. The organic phase is washed with a saturated sodium bicarbonate solution and then with water. The organic phase is dried and then concentrated under reduced pressure. The orange-colored oil obtained (2.6 g) is subjected to separation on a silica column (eluent:heptane/EtOAc 85:15). Clean fractions are recovered in the form of pale yellow flakes of the derivative of Example 3 (1.17 g, 40% yield):
UV (ethanol):
λ=370 nm, E1%=575; λₘₐₓ=342 nm, E1%=880; λ=300 nm, E1%=448.

### EXAMPLE 4

### Synthesis of 2,4-bis(dineopentyl 4′-aminobenzalmalonate)-6-(4″-amino-(tert-octyl)benzamide)-s-triazine of formula (4)

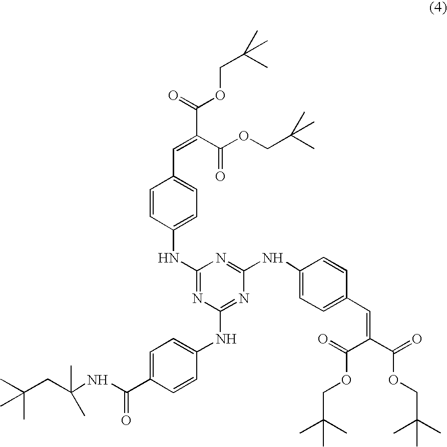

### First stage: Preparation of 4-nitro-N-(tert-octyl)benzamide

tert-Octylamine (51.7 g, 0.4 mol) and triethylamine (61.2 ml, 0.44 mol) are introduced into 260 ml of dichloroethane in a reactor. The mixture is heated to 70° C. and then 4-nitrobenzoyl chloride (77.9 g, 0.42 mol) is added over 50 minutes in small portions. The mixture is heated at reflux for 4 hours. The mixture is poured onto ice-cold water; extraction is carried out with dichloromethane, drying is carried out and the solvent is evaporated. The beige precipitate obtained is recrystallized from a mixture of isopropyl ether and of ethanol (ratio 10:1). After drying under vacuum, 84.6 g (76% yield) of 4-nitro-N-(tert-octyl)benzamide are obtained in the form of an off-white powder used as is in the following stage.

### Second Stage: Preparation of 4-amino-N-(tert-octyl)benzamide

4-Nitro-N-(tert-octyl)benzamide (30 g, 0.108 mol) dissolved in 200 ml of ethyl acetate is hydrogenated in a 500 ml hydrogenator in the presence of 4.8 g of 10% palladium-on-charcoal comprising 50% water as catalyst (hydrogen pressure: 8-10 bar) for 1 hour 15 minutes at a temperature of 70° C.-75° C. After filtering, concentrating the solvent and drying under vacuum, 20.4 g (yield: 76%) of 4-amino-N-(tert-octyl)benzamide are obtained in the form of a light yellow powder used as is in the following stage.

### Third Stage: Preparation of N-(tert-octyl)-4-[(4,6-dichloro-1,3,5-triazin-2-yl)amino]benzamide

Cyanuryl chloride (3.7 g, 0.0201 mol) is dissolved at 10° C. in 70 ml of dioxane in a reactor. A solution of the product from the preceding stage (5 g, 0.0201 mol) dissolved in 100 ml of dioxane and a solution of potassium carbonate (1.4 g, 0.03 mol) dissolved in 20 ml of water are added simultaneously thereto dropwise over 1 hour at 10° C. The heterogeneous mixture is left at a temperature of 10° C. for 2 hours. Approximately 300 ml of water are added and the precipitate formed is filtered off and then washed with water. After drying in a vacuum, 7.4 g (93% yield) of N-(tert-octyl)-4-[(4,6-dichloro-1,3,5-triazin-2-yl)amino]benzamide are obtained in the form of a white powder used as is in the following stage.

### Fourth Stage: Preparation of the Compound of Example 4

The intimately mixed mixture of the product from the preceding stage (0.29 g, 0.732×10⁻³ mol), of dineopentyl para-aminobenzalmalonate (0.5 g, 1.44×10⁻³ mol) and of sodium bicarbonate (0.14 g, 1.44×10⁻³ mol) is left in a CEM Discover microwave system for 4 minutes at a temperature of 60° C. and under a power of 300 watts and then for 15 minutes at a temperature of 110° C. The amorphous solid formed is extracted with dichloromethane. The organic phase is washed 3 times with water, is dried and is then concentrated under reduced pressure. The orange-colored oil obtained is subjected to separation on a silica column (eluent:heptane/EtOAc 75:25). Clean fractions are recovered in the form of a pale yellow oil which solidifies to give the derivative of Example 4 (0.6 g, 86% yield) in the form of a pale yellow powder:
UV (ethanol):
λₘₐₓ=351 nm, E1%=763; λ=298 nm, E1%=369.

### EXAMPLE 5

### Synthesis of 2,4-bis(dineopentyl 4′-aminobenzalmalonate)-6-(4″-amino-(tert-butyl)benzamide)-s-triazine of formula (5)

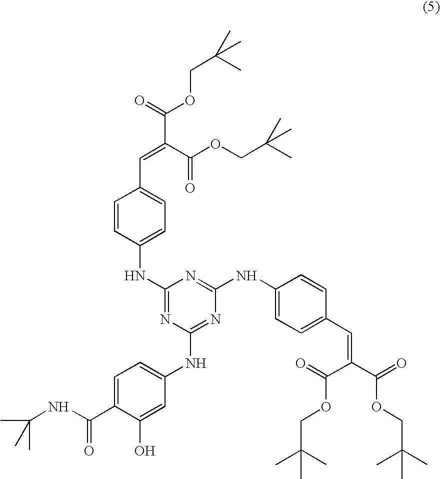

### First Stage: Preparation of N-(tert-butyl)-4-nitrobenzamide

4-Nitrobenzoyl chloride (18.9 g, 0.1 mol) dissolved in 60 ml of methylene chloride is added over 30 minutes at a temperature of 0-5° C. to a solution of tert-butylamine (8.3 g, 0.112 mol) and of triethylamine (15.6 ml, 0.112 mol) dissolved in 170 ml of dichloromethane in a reactor. The reaction mixture is brought back to laboratory temperature and left stirring for 2 hours. The organic phase is washed twice with water and dried. After removing the solvent under reduced pressure, the solid obtained is recrystallized from isopropanol. 17.1 g (yield: 77%) of N-(tert-butyl)-4-nitrobenzamide are obtained in the form of a pale yellow powder (M.p. 161-2° C.) used as is in the following stage.

### Second Stage: Preparation of 4-amino-N-(tert-butyl)benzamide

The preceding product (17.1 g, 0.077 mol), dissolved in 300 ml of isopropanol, is hydrogenated in a 1 liter hydrogenator in the presence of 3 g of 5% palladium-on-charcoal as catalyst (hydrogen pressure: 7 bar) for 30 minutes at a temperature of 60° C. After filtering, concentrating the solvent and drying under vacuum, 13.2 g (yield: 89%) of 4-amino-N-(tert-butyl)benzamide are obtained in the form of a light grey powder (M.p. 123-4° C.) used as is in the following stage.

### Third Stage: Preparation of N-(tert-butyl)-4-[(4,6-dichloro-1,3,5-triazin-2-yl)amino]benzamide

Cyanuryl chloride (11.71 g, 0.06 mol) is dissolved at 10° C. in 150 ml of dioxane in a reactor. A solution of the product from the preceding stage (11.53 g, 0.06 mol) dissolved in 60 ml of dioxane and a solution of potassium carbonate (6.3 g, 0.03 mol) dissolved in 30 ml of water are simultaneously added thereto dropwise at 10° C. over 1 hour. The heterogeneous mixture is left at a temperature of 10° C. for 2 hours. Approximately 300 ml of water are added and the precipitate formed is filtered off and then washed with water. After drying under vacuum, 18 g (88% yield) of N-(tert-butyl)-4-[(4,6-dichloro-1,3,5-triazin-2-yl)amino]benzamide are obtained in the form of a white powder (M.p. 256-7° C.) used as is in the following stage.

### Fourth Stage: Preparation of the Compound of Example 5

The intimately mixed mixture of the preceding product (1.5 g, 4.4×10⁻³ mol), of dineopentyl para-aminobenzalmalonate (3 g, 8.8×10⁻³ mol) and of sodium bicarbonate (0.37 g, 8.8×10⁻³ mol) is left in a CEM Discover microwave system for 4 minutes at a temperature of 60° C. and under a power of 300 watts and then for 20 minutes at a temperature of 150° C. The amorphous solid formed is extracted with dichloromethane. The organic phase is washed 3 times with water, is dried and is then concentrated under reduced pressure. The brown oil obtained is subjected to separation on a silica column (eluent:heptane/EtOAc 60:40). Clean fractions are recovered in the form of a pale yellow powder of the derivative of Example 5 (0.7 g, 17% yield):
UV (ethanol):
λ=375 nm, E1%=420; λₘₐₓ=345 nm, E1%=813; λ=299 nm, E1%=420.

EXAMPLE 6

### Synthesis of 2,4-bis(1,3-dimethylbutyl 4′-aminobenzalmalonate)-6-(amyl 4″-aminobenzoate)-s-triazine of formula (7)

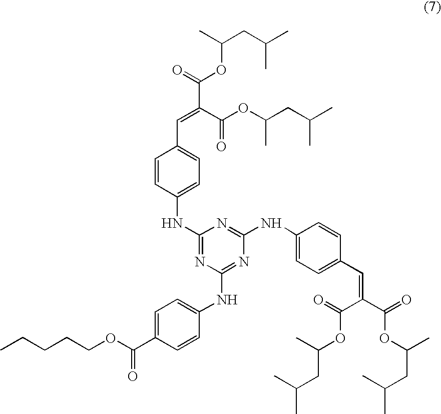

### First Stage: Preparation of 1,3-dimethylbutyl Malonate

Malonic acid (72.8 g, 0.7 mol) and the alcohol 2-methyl-4-pentanol (286 g, 2.8 mol) are brought to reflux for 2 hours in 200 ml of toluene in the presence of 1.8 ml of concentrated sulfuric acid in a reactor surmounted by a Dean & Stark apparatus. The water formed is removed by azeotropic distillation. The organic phase is washed 3 times with water and is dried over sodium sulfate. Filtration is carried out and the solvent is evaporated. The product obtained distils at 147° C. under 20 hPa. 160 g (79% yield) of 1,3-dimethylbutyl malonate are obtained in the form of a colorless oil which is used as is in the following stage.

### Second Stage: Preparation of 1,3-dimethylbutyl 4-nitrobenzalmalonate

p-Nitrobenzaldehyde (49.9 g, 0.33 mol) and 1,3-dimethylbutyl malonate (90 g, 0.33 mol) are placed in 150 ml of toluene in a round-bottomed flask equipped with a Dean & Stark apparatus surmounted by a reflux condenser and while sparging with nitrogen. The catalyst, prepared in advance, acetic acid (1.9 ml) and piperidine (3.3 ml) in suspension in 4 ml of toluene, is added thereto. The mixture is brought to reflux for 7 hours 30 minutes with stirring and the water formed is removed via the Dean & Stark apparatus. Two further additions of the same amounts of catalyst were necessary. The cooled reaction mixture is poured into water and extracted with dichloromethane. The organic phase is washed with water, then dried and concentrated under reduced pressure. The red-brown oil obtained is chromatographed on a silica column (eluent:heptane/EtOAc 97:3). 56.8 g (43% yield) of the clean fractions of 1,3-dimethylbutyl 4-nitrobenzalmalonate are recovered in the form of a yellow oil used as is in the following stage.

### Third Stage: Preparation of 1,3-dimethylbutyl 4-aminobenzalmalonate

The derivative from the preceding stage (56.8 g, 0.14 mol) is dispersed in 80 ml of acetic acid with stirring and while sparging with nitrogen. 115 ml of water are added thereto. The mixture is heated to 50° C. Iron (78.2 g, 1.4 mol) is added thereto portionwise without exceeding a temperature of 55° C. (time for introduction 1 hour). Subsequently, acetic acid (115 ml) is added dropwise without exceeding a temperature of 55° C. (time for introduction 2 hours). The mixture is heated for a further 1 hour at 55° C. It is cooled, water is added and extraction is carried out twice with dichloromethane. The organic phase is washed with water, with a saturated sodium bicarbonate solution and with water and is then dried over sodium sulfate. After concentrating under reduced pressure, a red-brown oil is obtained and is purified by passing through a silica column (eluent:heptane/EtOAc 85:15). It is recrystallized from a mixture of heptane and of 1,2-dichloroethane. 22.5 g (43% yield) of the clean fractions of 1,3-dimethylbutyl 4-aminobenzalmalonate are recovered in the form of an orange-colored oil used as is in the following stage.

### Fourth Stage: Preparation of the Compound of Example 6

The intimately mixed mixture of the product from the preceding stage (0.5 g, 1.32×10⁻³ mol), of the product from the second stage of Example 2 (0.235 g, 0.66×10⁻³ mol) and of sodium bicarbonate (0.11 g, 1.32×10⁻³ mol) is left in a CEM Discover microwave system for 4 minutes at a temperature of 60° C. and under a power of 300 watts and then for 25 minutes at a temperature of 110° C. The amorphous solid formed is extracted with dichloromethane. The organic phase is washed 3 times with water, is dried and is then concentrated under reduced pressure. The orange-colored oil obtained is subjected to separation on a silica column (eluent:heptane/EtOAc 80:20). Clean fractions are recovered in the form of a yellow oil which solidifies to give the derivative of Example 6 (0.4 g, 57% yield) in the form of light yellow flakes:
UV (ethanol):
λ=370 nm, E1%=500; λₘₐₓ=337 nm, E1%=800; λ=300 nm, E1%=411.

### EXAMPLE 7

### Synthesis of 2,4-bis(dimenthyl 4′-aminobenzalmalonate)-6-(amyl 4″-aminobenzoate)-s-triazine of formula (8)

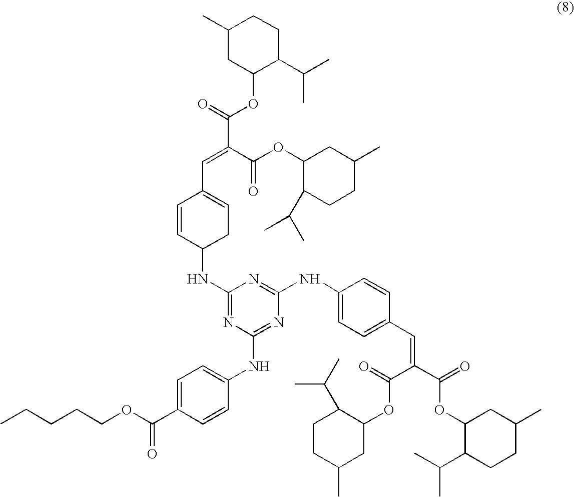

### First Stage: Preparation of Dimenthyl Malonate

Malonic acid (22.2 g, 0.213 mol) and menthol (70 g, 0.448 mol) are brought to reflux for 6 hours in 100 ml of toluene in the presence of 2 ml of concentrated sulfuric acid in a reactor surmounted by a Dean & Stark apparatus. The water formed is removed by azeotropic distillation. The organic phase is washed 3 times with water and is dried over sodium sulfate. The excess menthol is removed by distillation under vacuum (140° C. under 0.6 hPa). The residue is treated with animal charcoal in isopropanol at reflux. After filtering, rinsing and evaporating the solvent, 61.8 g (76% yield) of dimenthyl malonate are obtained in the form of a yellow oil which is used as is in the following stage.

### Second Stage: Preparation of dimenthyl 4-nitrobenzalmalonate

p-Nitrobenzaldehyde (21.8 g, 0.144 mol) and dimenthyl malonate (61 g, 0.16 mol) are placed in 100 ml of toluene in a round-bottomed flask equipped with a Dean & Stark apparatus surmounted by a reflux condenser and while sparging with nitrogen. The catalyst, prepared in advance, acetic acid (0.92 ml) and piperidine (1.6 ml) in suspension in 3 ml of toluene, is added thereto. The mixture is brought to reflux for 9 hours with stirring and the water formed is removed via the Dean & Stark apparatus. Three further additions of the same amounts of catalyst were necessary. The cooled reaction mixture is poured into water and extracted with dichloromethane. The organic phase is washed with water, then dried and concentrated under reduced pressure. The red brown oil obtained is chromatographed on a silica column (eluent: heptane/EtOAc 95:5). 37 g (50% yield) of the clean fractions of dimenthyl 4-nitrobenzalmalonate are recovered in the form of a yellow oil used as is in the following stage.

### Third Stage: Preparation of dimenthyl 4-aminobenzalmalonate

The derivative from the preceding stage (37 g, 0.072 mol) is dispersed in 30 ml of acetic acid and 45 ml of water with stirring and while sparging with argon. The mixture is heated to 50° C. Iron (24.4 g, 0.437 mol) is added thereto portionwise without exceeding a temperature of 55° C. (time for introduction 30 minutes). Subsequently, acetic acid (45 ml) is added dropwise without exceeding a temperature of 55° C. (time for introduction 1 hour 30 minutes). The mixture is heated for a further 1 hour at 55° C. It is cooled, water is added and extraction is carried out twice with dichloromethane. The organic phase is washed with water, with a saturated sodium bicarbonate solution and with water and is then dried over sodium sulfate. After concentrating under reduced pressure, an orange-colored gum is obtained and is purified by passing through a silica column (eluent:heptane/EtOAc 90:10). 8.6 g (25% yield) of the clean fractions of dimenthyl 4-aminobenzalmalonate are recovered in the form of a yellow solid used as is in the following stage.

### Fourth Stage: Preparation of the Compound of Example 7

The intimately mixed mixture of the preceding product (2 g, 4.1×10⁻³ mol), of 2,4-dichloro-6-(amyl 4′-aminobenzoate)-s-triazine (first stage of Example 2) (0.73 g, 2.05×10⁻³ mol) and of sodium bicarbonate (0.34 g, 4.1×10⁻³ mol) is left in a CEM Discover microwave system for 30 minutes at a temperature of 130-140° C. and under a power of 300 watts. The amorphous solid formed is extracted with dichloromethane. The organic phase is washed 3 times with water, is dried and is then concentrated under reduced pressure. The brown oil obtained is subjected to separation on a silica column (eluent:heptane/EtOAc 80:20). Clean fractions are recovered in the form of a pale yellow powder of the derivative of Example 7 (0.5 g, 16% yield):
UV (ethanol):
λ=370 nm, E1%=391; λₘₐₓ=340 nm, E1%=538; λ=300 nm, E1%=313.

### COMPOSITION EXAMPLES 8 TO 10

| | Ex. 8 | Ex. 9 | |
|---|---|---|---|
| | (outside | (outside | Ex. 10 |
| INGREDIENTS | the invention) | the invention) | (invention) |
| GLYCERYL STEARATE (and) | 1 | 1 | 1 |
| PEG-100 STEARATE | | | |
| SIMULSOL 165 (Seppic) | | | |
| STEARIC ACID | 1.5 | 1.5 | 1.5 |
| STEARINE TP 1200 | | | |
| (Stéarinerie Dubois) | | | |
| POLYDIMETHYLSILOXANE | 0.5 | 0.5 | 0.5 |
| 200 FLUID 350 CS (Dow | | | |
| Corning) | | | |
| CETYL ALCOHOL | 0.5 | 0.5 | 0.5 |
| NACOL 16-98 (Sasol) | | | |
| CETEARYL ALCOHOL (and) | 2 | 2 | 2 |
| CETEARYL GLUCOSIDE | | | |
| MONTANOV 68 (Seppic) | | | |
| PRESERVATIVE | 1 | 1 | 1 |
| TRIETHANOLAMINE | 0.45 | 0.45 | 0.45 |
| C₁₂₋₁₅ ALKYL BENZOATE | 15 | 15 | 15 |
| FINSOLV TN (Witco) | | | |
| 1,3,5- | — | — | 4 |
| TRIAZINE/BENZALMALONATE | | | |
| (EXAMPLE 1) | | | |
| BUTYL METHOXY | — | 2 | 2 |
| DIBENZOYLMETHANE | | | |
| PARSOL 1789 (DSM) | | | |
| ETHYLHEXYL | 5 | 5 | 5 |
| METHOXYCINNAMATE | | | |
| PARSOL MCX (DSM) | | | |
| GLYCEROL | 5 | 5 | 5 |
| PRICERINE 9091 (Uniquema) | | | |
| XANTHAN GUM | 0.2 | 0.2 | 0.2 |
| KELTROL T (NUTRASWEET) | | | |
| ETHYLENEDIAMINETETRAACETIC | 0.1 | 0.1 | 0.1 |
| ACID, DISODIUM SALT, | | | |
| 2H₂O (VERSENE NA2) | | | |
| POTASSIUM CETYL | 1 | 1 | 1 |
| PHOSPHATE | | | |
| AMPHISOL K (Givaudan) | | | |
| ACRYLATES/C₁₀₋₃₀ ALKYL | 0.2 | 0.2 | 0.2 |
| ACRYLATE CROSSPOLYMER | | | |
| PEMULEN TR-1 (Noveon) | | | |
| ISOHEXADECANE | 1 | 1 | 1 |
| ISOHEXADECANE (BP) | | | |
| TRIETHANOLAMINE | 0.2 | 0.2 | 0.2 |
| WATER | 70.35 | 63.35 | 59.3 |

Method for Measuring the Photostability:

For each formulation, constituents given as % by weight, 3 test samples and 3 control samples were prepared. 2 mg/cm² of formulation are deposited on poly(methyl methacrylate) sheets using a spatula.

The test sheets are exposed for 38 min to a Heraeus Suntest equipped with a xenon lamp having a UV-A flux of 9.51×10⁻³ W/cm² and a UV-B flux of 5.43×10⁻⁴ W/cm². The control sheets are stored for the same time and at the same temperature (38-40° C.) in darkness.

At the end of this time, the screening agents are extracted by immersing each sheet in 50 g of methanol and by subjecting them to ultrasound for 15 min in order to provide for good extraction. The solutions obtained are analyzed by HPLC and UV spectrophotometry.

For each formulation tested, the residual ethylhexyl methoxycinnamate level after exposure is given in the ratio of its optical density (OD) in the exposed sample to its unexposed optical density (OD). The positions adopted are the absorption maxima corresponding to ethylhexyl methoxycinnamate: λₘₐₓ=310 nm.

The results obtained are summarized in the following Table:

TABLE
Residual % of UV-B screening agent of the cinnamate type after 1
hour UV-A

| 5% | | |
|---|---|---|
| Cinnamate | | |
| UV-B | 5% Cinnamate UV-B screening agent and 2% | |
| screening | dibenzoylmethane UV-A screening agent | |
| agent alone | Composition 9 without | Composition 10 with |
| Composition 8 | triazine of formula (I) | triazine of formula (I) |
| 60% | 47% | 57% |

The loss under exposure of the UV-B screening agent of the cinnamic acid ester type is greatly accentuated in the presence of the UV-A screening agent of the dibenzoylmethane type.

The addition of the triazine of formula (I) makes it possible to improve the chemical stability of the UV-B screening agent and to maintain the balance in UV-A/UV-B protection.

Each patent, patent application, publication, text and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A topically applicable, photostable sunscreen composition containing at least one UV screening system, which comprises:
(a) at least one cinnamic acid ester UV-B screening agent,
(b) at least one dibenzoylmethane compound UV-A screening agent, and
(c) at least one s-triazine compound having the following formula (I): 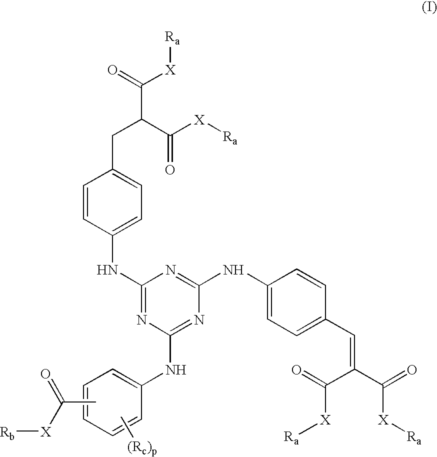
in which:
the groups X, which may be identical or different, are each —O— or —NR₆—;
the radicals Rₐ, which may be identical or different, are each a radical of formula (II): 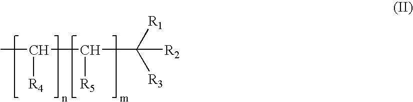
in which:
the radicals R₁ and R₂, which may be identical or different, are each a linear or branched C₁-C₈ alkyl radical, with the proviso that R₁ and R₂ may together form a C₅-C₈ ring member, optionally substituted by 1, 2 or 3 linear or branched C₁-C₄ alkyl radicals;
the radicals R₃, R₄ and R₅, which may be identical or different, are each a hydrogen atom or a linear or branched C₁-C₄ alkyl radical;
n has the value 0 or 1;
m has the value 0 or 1;
with the provisos that:
(i) when n=1 and R₄ is hydrogen, then m is equal to 0 and R₃ is other than hydrogen;
(ii) when R₁ and R₂ together form a C₅-C₈ ring member, then the sum n+m is other than 2;
R₆ is hydrogen or a C₁-C₈ alkyl radical;
R_{b} is a linear or branched and optionally unsaturated C₁-C₂₀ alkyl radical, a C₅-C₁₂ cycloalkyl radical optionally substituted by 1 to 3 linear or branched C₁-C₄ alkyl radicals, the —(CH₂CHR₇—O)_{q}R₈ radical or the —CH₂—CH(OH)—CH₂—O—R₈ radical;
R₇ is hydrogen or methyl;
R₈ is hydrogen or a C₁-C₈ alkyl radical;
q=1-20,
the COXR_{b} group can be in the ortho, meta or para position with respect to the amino group;
R_{c} is a saturated or unsaturated and linear or branched C₁-C₂₀ alkyl radical, the OH group or a linear or branched C₁-C₂₀ alkoxy radical; and
p is equal to 0, 1 or 2, formulated into (d) a topically applicable, cosmetically acceptable medium therefor.

2. The photostable sunscreen composition as defined by claim 1, wherein formula (I):
(a) n=m=0 and
(b) R₁, R₂, R₃ are each a C₁-C₄ alkyl radical or R₃ is hydrogen and R₁ and R₂ together form a C₅-C₈ ring member optionally substituted by one or two alkyl radicals.

3. The photostable sunscreen composition as defined by claim 1, wherein formula (I):
(a) n=1 and R₄ is an alkyl radical or m=1 and R₅ is an alkyl radical, and
(b) R₁ and R₂ are each a C₁-C₄ alkyl radical.

4. The photostable sunscreen composition as defined by claim 1, wherein the compounds of formula (I) are selected from the group consisting of the compounds of following formulae (1) to (10):

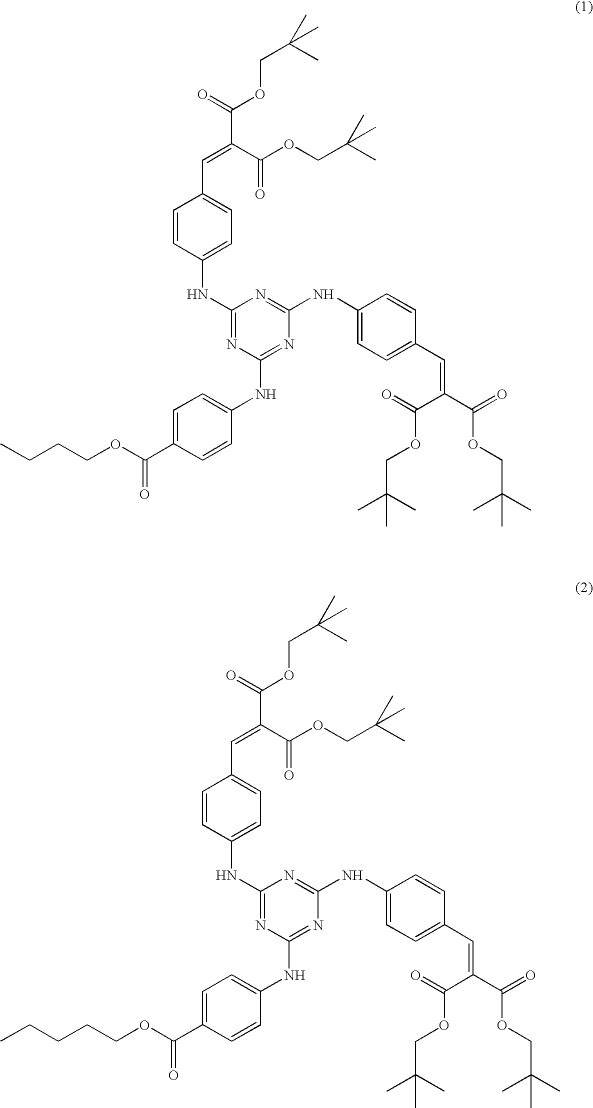

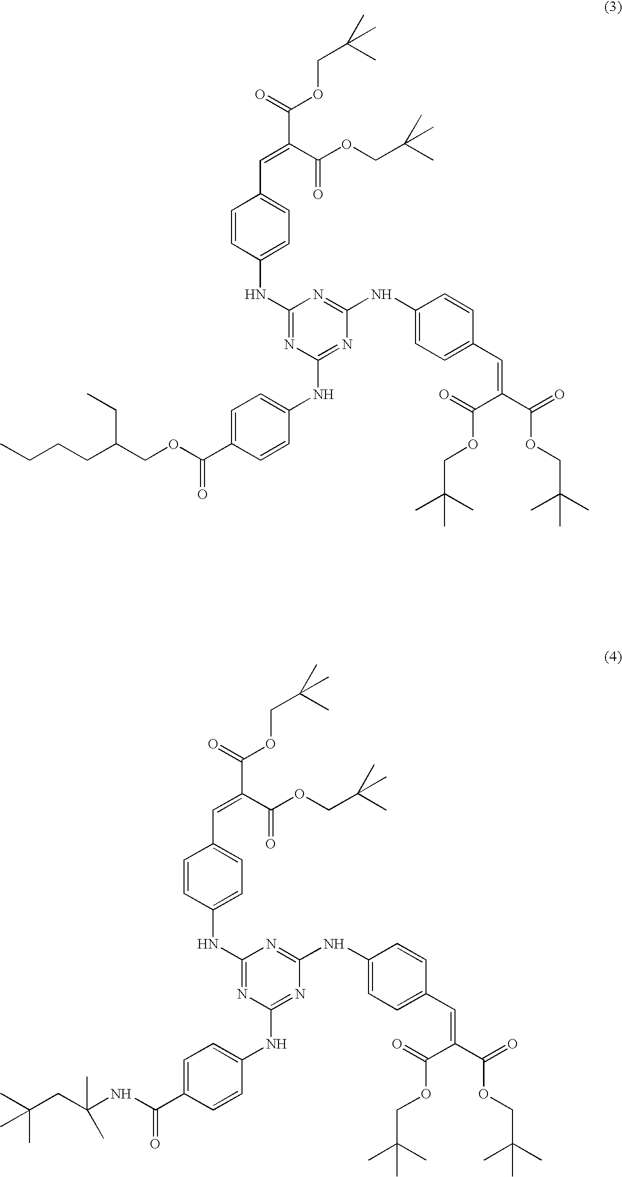

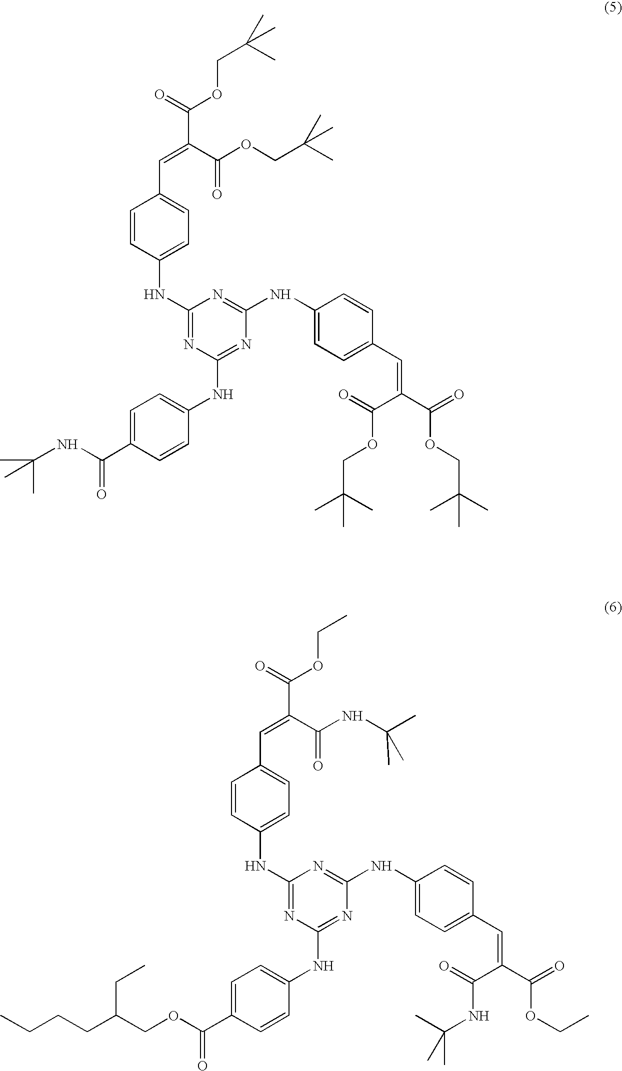

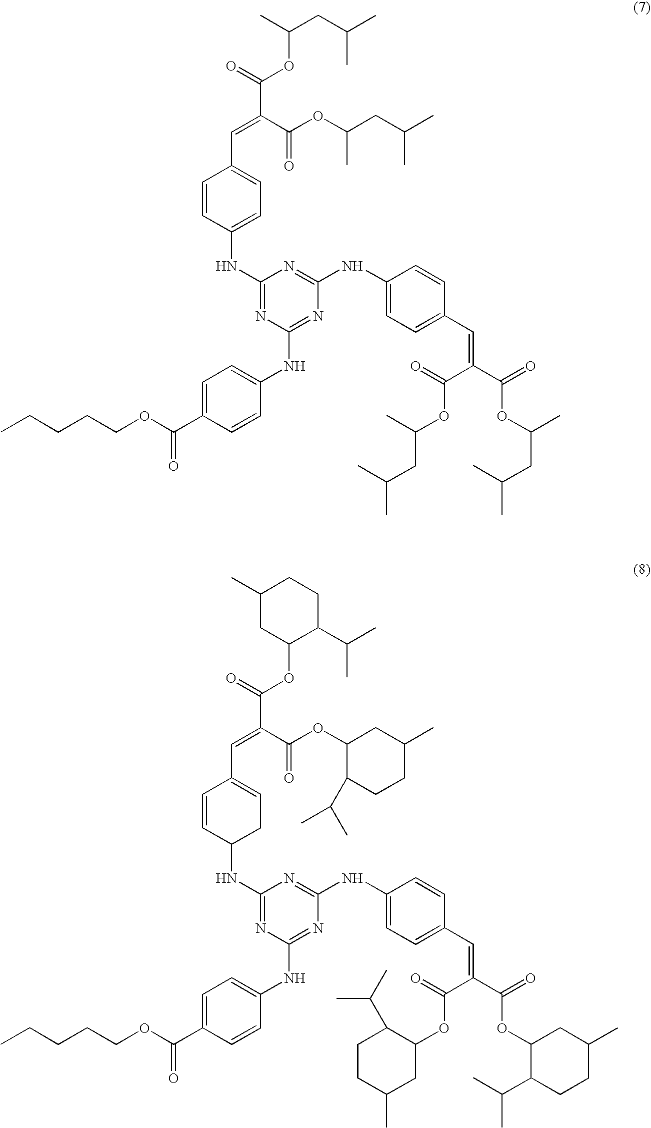

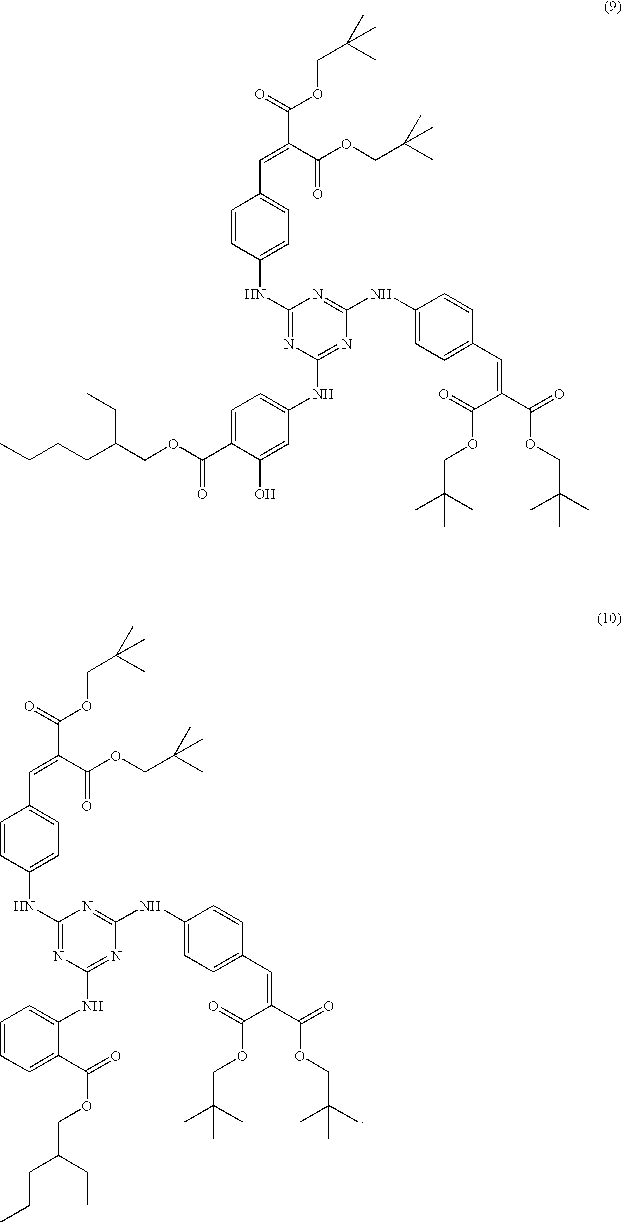

5. The photostable sunscreen composition as defined by claim 4, wherein the compound of formula (I) comprises 2,4-bis(dineopentyl 4′-aminobenzalmalonate)-6-(butyl 4″-aminobenzoate)-s-triazine of formula (1).

6. The photostable sunscreen composition as defined by claim 1, said at least one cinnamic acid ester UV-B screening agent having the following formula (A): 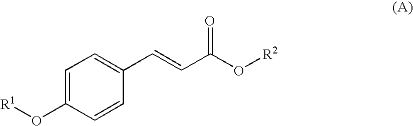
in which:
the radicals R¹ and R², which may be identical or different, are each a linear or branched C₁-C₂₄ alkyl radical.

7. The photostable sunscreen composition as defined by claim 6, said at least one cinnamic acid ester UV-B screening agent being selected from the group consisting of:
Ethylhexyl Methoxycinnamate or Cinoxate,
Isopropyl Methoxycinnamate,
Isoamyl Methoxycinnamate, and
Diisopropyl Methylcinnamate.

8. The photostable sunscreen composition as defined by claim 7, said at least one cinnamic acid ester UV-B screening agent comprising Ethylhexyl Methoxycinnamate or Cinoxate.

9. The photostable sunscreen composition as defined by claim 1, said at least one cinnamic acid ester UV-B screening agent comprising from 0.01% to 20% by weight thereof, with respect to the total weight of the composition.

10. The photostable sunscreen composition as defined by claim 1, said at least one dibenzoylmethane compound UV-A screening agent being selected from the group consisting of:
2-methyldibenzoylmethane,
4-methyldibenzoylmethane,
4-isopropyldibenzoylmethane,
4-(tert-butyl)dibenzoylmethane,
2,4-dimethyldibenzoylmethane,
2,5-dimethyldibenzoylmethane,
4,4′-diisopropyldibenzoylmethane,
4,4′-dimethoxydibenzoylmethane,
4-(tert-butyl)-4′-methoxydibenzoylmethane,
2-methyl-5-isopropyl-4′-methoxydibenzoylmethane,
2-methyl-5-(tert-butyl)-4′-methoxydibenzoylmethane,
2,4-dimethyl-4′-methoxydibenzoylmethane, and
2,6-dimethyl-4-(tert-butyl)-4′-methoxydibenzoylmethane.

11. The photostable sunscreen composition as defined by claim 10, said at least one dibenzoylmethane compound UV-A screening agent comprising 4-isopropyldibenzoylmethane, having the following formula:

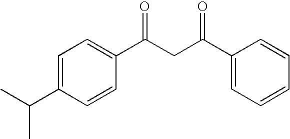

12. The photostable sunscreen composition as defined by claim 10, said at least one dibenzoylmethane compound UV-A screening agent comprising 4-(tert-butyl)-4′-methoxydibenzoylmethane or Butyl Methoxy Dibenzoylmethane, having the following formula:

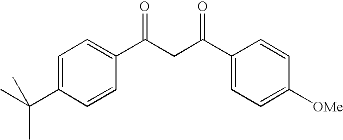

13. The photostable sunscreen composition as defined by claim 1, said at least one dibenzoylmethane compound UV-A screening agent comprising from 0.01% to 20% by weight thereof, with respect to the total weight of the composition.

14. The photostable sunscreen composition as defined by claim 1, said at least one compound of formula (I) comprising from 0.01% to 20% by weight thereof, with respect to the total weight of the composition.

15. The photostable sunscreen composition as defined by claim 1, further comprising at least one other organic or inorganic photoprotective agent active in the UV-A and/or UV-B region which is water-soluble or fat-soluble or insoluble in the conventional cosmetic solvents.

16. The photostable sunscreen composition as defined by claim 15, comprising at least one additional organic photoprotective agent selected from the group consisting of anthranilates; salicylic derivatives; camphor derivatives; benzophenone derivatives; β,β-diphenylacrylate derivatives; triazine derivatives other than those of formula (I); benzotriazole derivatives; benzalmalonate derivatives other than those of formula (I); benzimidazole derivatives; imidazolines; bis-benzoazolyl derivatives; p-aminobenzoic acid (PABA) derivatives other than those of formula (I); methylenebis(hydroxyphenylbenzotriazole) derivatives; benzoxazole derivatives; screening polymers and screening silicones; dimers derived from α-alkylstyrene; 4,4-diarylbutadienes; and mixtures thereof.

17. The photostable sunscreen composition as defined by claim 16, comprising at least one of the following compounds:
Ethylhexyl Salicylate,
Homosalate,
Octocrylene,
Phenylbenzimidazole Sulfonic Acid,
Disodium Phenyl Dibenzimidazole Tetrasulfonate,
Benzophenone-3,
Benzophenone-4,
Benzophenone-5,
Diethylamino Hydroxybenzoyl Hexyl Benzoate,
4-Methylbenzylidene Camphor,
Terephthalylidene Dicamphor Sulfonic Acid,
Bis-Ethylhexyloxyphenol Methoxyphenyl Triazine,
Ethylhexyl Triazone,
Diethylhexyl Butamido Triazone,
Methylene Bis-Benzotriazolyl Tetramethylbutylphenol,
Drometrizole Trisiloxane,
Polysilicone-15,
1,1-Dicarboxy(2,2′-Dimethylpropyl)-4,4-Diphenylbutadiene, and and mixtures thereof.

18. The photostable sunscreen composition as defined by claim 15, comprising at least one additional inorganic photoprotective agent selected from the group consisting of pigments or nanopigments formed of metal oxides, whether treated or untreated.

19. The photostable sunscreen composition as defined by claim 18, comprising pigments or nanopigments of titanium, zinc, iron, zirconium or cerium oxides and mixtures thereof, whether treated or untreated.

20. The photostable sunscreen composition as defined by claim 1, further comprising at least one agent for the artificial tanning and/or browning of the skin.

21. The photostable sunscreen composition as defined by claim 1, further comprising at least one adjuvant selected from the group consisting of fatty substances, organic solvents, ionic or nonionic and hydrophilic or lipophilic thickeners, softening agents, humectants, opacifiers, stabilizing agents, emollients, silicones, anti-foaming agents, fragrances, preservatives, anionic, cationic, nonionic, zwitterionic or amphoteric surfactants, active principles, fillers, polymers, propellants, basifying or acidifying agents, and mixtures thereof.

22. The photostable sunscreen composition as defined by claim 1, formulated as an oil-in-water or water-in-oil emulsion.

23. The photostable sunscreen composition as defined by claim 1, formulated as a product for the cosmetic treatment of the skin, lips, nails, hair, eyelashes, eyebrows and/or scalp.

24. The photostable sunscreen composition as defined by claim 1, formulated as a care product for the skin, lips, nails, hair and/or scalp.

25. The photostable sunscreen composition as defined by claim 1, formulated as a makeup product.

26. A method for improving the chemical stability with respect to UV radiation of at least one cinnamic acid ester UV-B screening agent in the presence of a dibenzoylmethane compound UV-A screening agent which comprises combining, with this mixture of UV screening agents, at least one s-triazine compound of formula (I) as defined in claim 1.

27. A method for improving the chemical stability of at least one cinnamic acid ester UV-B screening agent with respect to UV radiation, comprising formulating at least one s-triazine compound of formula (I) as defined in claim 1 in a composition which comprises at least one cinnamic acid ester UV-B screening agent and at least one dibenzoylmethane compound UV-A screening agent.

28. A method of improving the effectiveness of the following composition with respect to UV-B rays, comprising formulating at least one s-triazine compound of formula (I) as defined in claim 1 in a composition which comprises at least one cinnamic acid ester UV-B screening agent and at least one dibenzoylmethane compound UV-A screening agent.

29. A method of maintaining the balance in UV-A/UV-B protection during exposure to the sun of the following composition, comprising formulating at least one s-triazine compound of formula (I) as defined in claim 1 in a composition which comprises at least one cinnamic acid ester UV-B screening agent and at least one dibenzoylmethane compound UV-A screening agent.

30. A regime or regimen for photoprotecting the skin, lips, nails, hair, eyelashes, eyebrows, and/or scalp against the damaging effects of UV radiation, comprising topically applying thereon a thus effective amount of the photostable sunscreen composition as defined in claim 1.
